(19) United States
(12) Patent Application Publication
(10) Pub. No.: US 2025/0170139 A1
(43) Pub. Date: May 29, 2025

(54) AZASETRON FOR THE TREATMENT OF SUDDEN SENSORINEURAL HEARING LOSS

(71) Applicant: SENSORION, Montpellier (FR)

(72) Inventors: Judith LAREDO, Montpellier (FR); Géraldine HONNET, Montpellier (FR)

(73) Assignee: SENSORION, Montpellier (FR)

(21) Appl. No.: 18/840,560

(22) PCT Filed: Mar. 16, 2023
(86) PCT No.: PCT/EP2023/056761

§ 371 Date: Aug. 22, 2024

(30) Foreign Application Priority Data
Mar. 16, 2022 (EP) 22305311.7

(51) Int. Cl. A61K 31/538; A61K 31/573; A61P 27/16

(52) U.S. Cl. CPC A61K 31/538; A61K 31/573; A61P 27/16

(57) ABSTRACT

Azasetron or an analog thereof, or a pharmaceutically acceptable salt and/or solvate thereof, for treating severe or profound sudden sensorineural hearing loss (SSNHL) in a subject in need thereof. In particular, azasetron or an analog thereof, or a pharmaceutically acceptable salt and/or solvate thereof, for treating severe or profound SSNHL in a subject suffering from severe or profound SSNHL associated with (i) a hearing threshold at baseline corresponding to a pure tone audiometry (PTA) at baseline equal to or greater than 70 dB, preferably equal to or greater than 80 dB, more preferably equal to or greater than 90 dB; (ii) a hearing loss at baseline equal to or greater than 91 dB corresponding to a profound hearing loss according to the American Speech-Language-Hearing Association (ASHA) classification; (iii) a hearing loss at baseline affecting frequencies equal to or lower than 2000 Hz; and/or (iv) the presence of vertigo at baseline.

### FIELD OF INVENTION

The present invention relates to the treatment of sudden sensorineural hearing loss (SSNHL), in particular of severe or profound SSNHL.

### BACKGROUND OF INVENTION

Sudden sensorineural hearing loss (SSNHL), commonly known as sudden deafness, refers to a rapid-onset subjective sensation of hearing impairment. SSNHL is more particularly defined as a decrease in hearing of 30 dB or greater over at least three contiguous audiometric frequencies occurring within a 72-hour period. In nearly all subjects, SSNHL is unilateral and only affects one ear. Indeed, less than 2% of subjects suffering from SSNHL have bilateral involvement and typically bilateral involvement is sequential. An estimated maximum of 32% to 65% of cases of SSNHL may recover spontaneously. However, clinical experience indicates that this recovery rate may be an overestimation. Moreover, while recovery of hearing thresholds following SSNHL may be complete, it may also be partial and may frequently not occur at all.

SSNHL is a rare condition with an average total annual incidence rate of 4 to 160 per 100,000. However, the true incidence of SSNHL may be higher because affected subjects who recover quickly may not present for medical care. Approximately 66,600 new cases are reported each year in the United States of America. The incidence increases with age, ranging from 11 per 100,000 for patients younger than 18 years to 77 per 100,000 for patients 65 years and older (Alexander et al. Incidence of sudden sensorineural hearing loss. Otol Neurotol. 2013 December; 34 (9): 1586-9). The most commonly reported causes of SSNHL include infectious and otologic diseases; trauma, including for example noise-induced trauma, barotrauma and head trauma; vascular/hematologic causes including for example cerebrovascular accidents; and neoplastic causes including for example schwannomas and meningiomas. However, in the majority of subjects, SSNHL does not have an identifiable cause. In these subjects, SSNHL is classified as idiopathic.

SSNHL is a serious and debilitating condition. Hearing loss adversely affects communication, speech perception in noisy environments, and social interaction, substantiating that unilateral deafness is a serious disability. Disorientation and confusion from the inability to localize sound may put subjects at risk for accidents and hearing loss is associated with significantly increased odds of falling in older adults. A psychosocial impact was reported in almost 75% of subjects who had even mild unilateral hearing loss, and feeling frustrated, upset, and left out were commonly reported.

There is currently no approved treatment to improve or facilitate full, or even partial, recovery of hearing and/or symptoms of SSNHL, regardless of the cause. The treatment of SSNHL thus remains somewhat controversial, and is widely variable and often regionally specific. The American Academy of Otolaryngology-Head and Neck Surgery (AAO-HNS) has issued in 2012 clinical practice guidelines for the management of SSNHL. No European medical society has published such guidelines. In clinical practice, corticosteroids are often administered, either systemically and/or intratympanically. A large number of other treatments, including for example antivirals, antibiotics, diuretics, vasodilators, osmotic agents, plasma expanders, anticoagulants, mineral supplements, and hyperbaric oxygen or carbon dioxide-rich gases, have been used and/or evaluated without consistent success. Recent meta-analyses evaluating the efficacy and safety of medical interventions for the treatment of SSNHL have not identified any effective therapy (Wei et al. Steroids for idiopathic sudden sensorineural hearing loss. Cochrane Database Syst Rev. 2013 Jul. 2; 2013 (7): CD003998; Lawrence et al. Controversies in the management of sudden sensorineural hearing loss: an evidence-based review. Clin Otolaryngol. 2015 June; 40 (3): 176-82).

Therefore, there is still a need for an effective treatment for SSNHL, in particular for severe or profound SSNHL. It may thus be helpful to identify the subjects suffering from SSNHL, in particular severe or profound SSNHL, most likely to benefit from treatment.

The Inventors surprisingly showed that (R)-azasetron significantly induces a hearing improvement in subjects suffering from severe or profound SSNHL associated with at least one of (i) a hearing threshold at baseline corresponding to a pure tone audiometry (PTA) at baseline equal to or greater than 70 dB; (ii) a hearing loss at baseline equal to or greater than 91 dB corresponding to a profound hearing loss according to the American Speech-Language-Hearing Association (ASHA) classification; (iii) a hearing loss at baseline affecting frequencies equal to or lower than 2000 Hz; and/or (iv) the presence of vertigo at baseline.

The present invention thus relates to azasetron or an analog of azasetron, or a pharmaceutically acceptable salt and/or solvate thereof, for use in the treatment of severe or profound SSNHL in a subject in need thereof, in particular in a subject most likely to benefit from the administration of azasetron or an analog of azasetron, or a pharmaceutically acceptable salt and/or solvate thereof, such as a subject having (i) a hearing threshold at baseline corresponding to a PTA at baseline equal to or greater than 70 dB, preferably equal to or greater than 80 dB, more preferably equal to or greater than 90 dB; (ii) a hearing loss at baseline equal to or greater than 91 dB corresponding to a profound hearing loss according to the ASHA classification; (iii) a hearing loss at baseline affecting frequencies equal to or lower than 2000 Hz; and/or (iv) vertigo at baseline.

### SUMMARY

The present invention relates to azasetron or an analog of azasetron, or a pharmaceutically acceptable salt and/or solvate thereof, for use in the treatment of sudden sensorineural hearing loss (SSNHL) in a subject in need thereof, wherein the subject suffers from severe or profound SSNHL associated with at least one of:
a hearing threshold at baseline corresponding to a pure tone audiometry (PTA) at baseline equal to or greater than 70 dB, preferably equal to or greater than 80 dB, more preferably equal to or greater than 90 dB;
a hearing loss at baseline equal to or greater than 91 dB corresponding to a profound hearing loss according to the American Speech-Language-Hearing Association (ASHA) classification;
a hearing loss at baseline affecting frequencies equal to or lower than 2000 Hz; and/or
the presence of vertigo at baseline.

In some embodiments, the subject suffers from severe or profound SSNHL associated with a hearing threshold at baseline corresponding to a PTA at baseline equal to or greater than 80 dB. In some embodiments, the subject suffers from severe or profound SSNHL associated with a hearing threshold at baseline corresponding to a PTA at baseline equal to or greater than 90 dB. In some embodiments, the subject suffers from severe or profound SSNHL associated with a hearing loss at baseline affecting frequencies equal to or lower than 2000 Hz.

In some embodiments, SSNHL is idiopathic SSNHL. In some embodiments, SSNHL is unilateral.

In some embodiments, azasetron is (R)-azasetron, (S)-azasetron, a mixture thereof, or a pharmaceutically acceptable salt and/or solvate thereof; preferably azasetron is (R)-azasetron or a pharmaceutically acceptable salt and/or solvate thereof. In some embodiments, the pharmaceutically acceptable salt is selected from the group comprising or consisting of a besylate salt, a malate salt, and a hydrochloride salt. In some embodiments, the pharmaceutically acceptable salt is (R)-azasetron besylate. In some embodiments, the analog of azasetron is a benzoxazine compound or a pharmaceutically acceptable salt and/or solvate thereof, preferably selected from the group comprising or consisting of 6-chloro-3,4-dihydro-2-methyl-3-oxo-N-(3-quinuclidinyl)-2H-1,4-benzoxazine-8-carboxamide, 6-chloro-3,4-dihydro-2,4-dimethyl-3-oxo-N-(3-quinuclidinyl)-2H-benzoxazine-8-carboxamide, 6-chloro-2-ethyl-3,4-dihydro-4-methyl-3-oxo-N-(3-quinuclidinyl)-2H-1,4-benzoxazine-8-carboxamide, 6-chloro-3,4-dihydro-4-methyl-3-oxo-N-(3-quinuclidinyl)-2H-1,4-benzoxazine-8-carboxamide, 6-bromo-3,4-dihydro-2,4-dimethyl-3-oxo-N-(3-quinuclidinyl)-2H-1,4-benzoxazine-8-carboxamide, 6-chloro-3,4-dihydro-2,2,4-trimethyl-3-oxo-N-(3-quinuclidiny-1)-2H-1,4-benzoxazine-8-carboxamide, and pharmaceutically acceptable salts or solvates thereof.

In some embodiments, azasetron or the analog of azasetron, or the pharmaceutically acceptable salt and/or solvate thereof, is to be administered at a daily dose ranging from about 20 mg to about 200 mg. In some embodiments, azasetron or the analog of azasetron, or the pharmaceutically acceptable salt and/or solvate thereof, is to be administered at a daily dose of about 40 mg or of about 60 mg.

In some embodiments, azasetron or the analog of azasetron, or the pharmaceutically acceptable salt and/or solvate thereof, is to be administered within about 96 hours from the first onset of SSNHL.

In some embodiments, azasetron or the analog of azasetron, or the pharmaceutically acceptable salt and/or solvate thereof, is to be administered with at least one further pharmaceutically active agent. In some embodiments, the at least one further pharmaceutically active agent is a corticosteroid.

### Definitions

In the present invention, the following terms have the following meanings:

“SSNHL” refers to sudden sensorineural hearing loss.

“About”, when preceding a figure, encompasses plus or minus 10%, or less, of the value of said figure. It is to be understood that the value to which the term “about” refers is itself also specifically, and preferably, disclosed.

“Baseline”, when qualifying a parameter observed in a subject suffering from SSNHL, in particular severe or profound SSNHL, refers to the time preceding the start of administration to the subject of azasetron or an analog of azasetron, or a pharmaceutically acceptable salt and/or solvate thereof, as described herein. For example, for a given subject, the hearing loss at baseline is the hearing loss of the subject prior to the administration to the subject of azasetron or an analog of azasetron, or a pharmaceutically acceptable salt and/or solvate thereof, as described herein.

“Pharmaceutically acceptable excipient” or “pharmaceutically acceptable carrier” refers to an excipient or carrier that does not produce an adverse, allergic or other untoward reaction when administered to a subject. It includes any and all solvents, such as, for example, dispersion media, coatings, antibacterial and antifungal agents, isotonic and absorption delaying agents. A pharmaceutically acceptable excipient or carrier refers to a non-toxic solid, semi-solid or liquid filler, diluent, encapsulating material or formulation auxiliary of any type. For human administration, preparations should meet sterility, pyrogenicity, general safety and purity standards as required by the regulatory offices such as the FDA (U.S. food and drug administration) or EMA (European medicines agency).

“Subject” refers to a mammal, preferably a human. In some embodiments, the mammal is selected from cats, dogs, cows, pigs, horses, monkeys, apes and humans. In some embodiments, the mammal is selected from cats, dogs and humans. In some embodiments, the mammal is a primate. In some embodiments, the mammal is a human. In some embodiments, the subject may be a “patient”, i.e., a mammal, preferably a human, who/which is awaiting the receipt of, or is receiving medical care or was/is/will be the object of a medical procedure, or is monitored for SSNHL, in particular severe or profound SSNHL. In some embodiments, a “subject in need of treatment” is a subject who is awaiting the receipt of, or is receiving medical care, or was/is/will be the object of a medical procedure, or is monitored for SSNHL, in particular severe or profound SSNHL.

“Therapeutically effective amount” or “therapeutically effective dose” refers to the amount or dose of azasetron or an analog of azasetron, or a pharmaceutically acceptable salt and/or solvate thereof, that is aimed at, without causing significant negative or adverse side effects to the subject in need of treatment, bringing about at least one of the following: (1) slowing down or stopping the progression, aggravation, or deterioration of one or more symptom(s) of SSNHL, in particular severe or profound SSNHL; (2) bringing about ameliorations of one or more symptom(s) of SSNHL, in particular severe or profound SSNHL; (3) reducing the severity of SSNHL, in particular severe or profound SSNHL; (4) curing SSNHL, in particular severe or profound SSNHL, and/or (5) increasing quality of life. Examples of symptoms of SSNHL include hearing loss and tinnitus. In some embodiments, a therapeutically effective amount or dose is an amount or dose of azasetron or an analog of azasetron, or a pharmaceutically acceptable salt and/or solvate thereof, that is aimed at, without causing significant negative or adverse side effects to the subject in need of treatment, bringing about a hearing improvement. For example, a hearing improvement may correspond to a decrease of the hearing loss; an improvement in the hearing threshold, in particular an improvement in the hearing threshold corresponding to an improvement in pure tone audiometry (PTA); and/or an improvement in word recognition score. In some embodiments, an improvement in PTA is defined as an improvement in the most affected ear of a least about 30, 35, 40, 45, 50, or 55 dB, preferably of at least about 40 dB, as compared to the PTA at baseline (i.e., PTA before administration of azasetron or an analog of azasetron, or a pharmaceutically acceptable salt and/or solvate thereof). In some embodiments, an improvement in word recognition score is defined as an improvement of at least about 15% as compared to the word recognition score at baseline at 60 dB or 80 dB (i.e., word recognition score before administration of azasetron or an analog of azasetron, or a pharmaceutically acceptable salt and/or solvate thereof).

“Treating” or “Treatment” refers to a therapeutic treatment, to a prophylactic (or preventative) treatment, or to both a therapeutic treatment and a prophylactic (or preventative) treatment, wherein the object is to prevent, reduce, or slow down (lessen) one or more of the symptom(s) or manifestation(s) of SSNHL, in particular severe or profound SSNHL. In some embodiments, a subject is successfully “treated” for SSNHL, in particular severe or profound SSNHL, if, after receiving a therapeutically effective amount or dose of azasetron or an analog of azasetron, or a pharmaceutically acceptable salt and/or solvate thereof, the subject shows a hearing improvement, for example as defined herein. Parameters for assessing successful treatment, in particular hearing improvement, are readily measurable by routine procedures familiar to a physician. Examples of methods for measuring hearing loss, also useful for assessing hearing improvement, are mentioned hereinafter.

### DETAILED DESCRIPTION

The present invention relates to azasetron or an analog of azasetron, or a pharmaceutically acceptable salt and/or solvate thereof, for use in the treatment of sudden sensorineural hearing loss (SSNHL) in a subject in need thereof. In some embodiments, SSNHL is severe or profound SSNHL. Thus, in some embodiments, the subject in need of treatment is a subject suffering from severe or profound SSNHL.

Sudden sensorineural hearing loss (SSNHL) is a subset of sudden hearing loss. SSNHL is characterized by an impairment or damage of the cochlea, auditory nerve, or other aspects of central auditory perception or processing. SSNHL may be defined by the following audiometric criterion: a decrease in hearing (that is to say a hearing loss) of at least about 10, 20, or 30 decibels (dB), or more, occurring within about 72 hours or less. In some embodiments, SSNHL is defined as a decrease in hearing (that is to say a hearing loss) of at least about 10, 20, 30 decibels (dB) or more over at least three contiguous frequencies, occurring within about 72 hours or less.

Hearing loss is commonly established by comparison to a reference value, preferably resulting from previously obtained audiometry (i.e., obtained before onset of SSNHL). When previously obtained audiometry (i.e., audiometry before onset of SSNHL) is not available, hearing loss may be established by comparison to a reference value derived from a reference population. For unilateral SSNHL, hearing loss may be defined by comparison to the unaffected hear.

Methods for measuring hearing loss are well-known by the skilled artisan. Examples of such methods include pure tone audiometry (PTA) corresponding to pure tone audiometric air conduction test and/or pure tone audiometric bone conduction test, speech audiometry, behavioral observation audiometry, visual reinforcement audiometry, conditioned play audiometry, ABR (auditory brainstem responses) measurement, DPOAE (distortion product otoacoustic emissions) measurement, TEOAE (transiently evoked otoacoustic emissions) measurement, speech in noise test, word comprehension test, tympanometry, acoustic reflex tests, and tuning fork test.

In some embodiments, SSNHL is defined as a hearing loss of about 30 dB or more, corresponding to the difference between a PTA (preferably pure tone audiometric air conduction testing) expressed as the average of the hearing threshold (in dB) at the 3 most affected contiguous test frequencies and a reference PTA (preferably pure tone audiometric air conduction testing) expressed as the average of the hearing threshold for the same frequencies.

In some embodiments, SSNHL is defined as:
a hearing loss of about 30 dB or more, corresponding to the difference between a PTA (preferably pure tone audiometric air conduction testing) expressed as the average of the hearing threshold (in dB) at the 3 most affected contiguous test frequencies and a reference PTA (preferably pure tone audiometric air conduction testing) expressed as the average of the hearing threshold for the same frequencies, and
at least one hearing threshold of 50 dB or more amongst the 3 most affected contiguous frequencies on PTA, preferably on pure tone audiometric air conduction testing.

In some embodiments, the reference PTA is a PTA of the unaffected contralateral ear, a previously obtained PTA (i.e., a PTA obtained before onset of SSNHL), or a reference PTA derived from a reference population.

Pure tone audiometry (PTA) is widely used to assess SSNHL. The primary purpose of PTA is to determine the degree, type and configuration of hearing loss as its measurement covers the entire auditory pathway from the external ear to the cortex. PTA is a standardized subjective hearing test determining air-conduction and/or bone-conduction hearing thresholds (expressed in dB) at a set of fixed frequencies ranging from 0.25 kHz to 8 kHz. The hearing thresholds determined at each frequency are plotted on an audiogram for each ear independently. For example, the hearing threshold may be defined as the lowest audible level, measured twice out of three presentations to the subject. Thus, PTA allows to determine a hearing threshold.

In some embodiments, the PTA is expressed as the average of the hearing threshold (in dB) of the 3 most affected contiguous test frequencies. In some embodiments, the PTA is expressed as the average of the hearing threshold (in dB) of the 3 most affected contiguous test frequencies, using pure tone air-conduction testing.

For example, pure tone air-conduction may be determined at 0.25, 0.5, 1, 2, 3, 4, 6, and 8 kHz. For example, pure tone bone-conduction hearing thresholds may be determined at 0.5, 1, 2, 3, 4 kHz. Testing may be carried out using the descending method of limits (modified Hughson Westlake procedure as follows: initial descent towards threshold is accomplished in 10-dB steps; beginning with the first nonresponse, level is increased by 5-dB for each non-response and decreased by 10-dB after each correct detection response).

As indicated above, the hearing of a subject (including hearing loss and/or hearing improvement) may be assessed with speech audiometry. Speech audiometry is a diagnostic hearing test designed to test word or speech recognition. It has become a fundamental tool in hearing-loss assessment. In conjunction with PTA, it can aid in determining the degree and type of hearing loss. Speech audiometry also provides information regarding discomfort or tolerance to speech stimuli and information on word recognition abilities.

Speech audiometry includes for example speech recognition assessed by determining the speech recognition threshold, and word recognition assessed by determining the word recognition score.

The speech recognition threshold is the minimum hearing level (in dB) at which the subject can correctly recognize 50% of the presented speech material. Spondaic words are the usual and recommended test material for determining the speech recognition threshold. Spondaic words are two-syllable words with equal stress on both syllables (e.g., “birthday”). According to the ASHA (American Speech-Language-Hearing Association) guidelines, subjects are familiarized with the spondaic words prior to the test. Subjects listen to the list of words and indicate if any are unfamiliar, so that they may be eliminated from the list. The median speech reception threshold (SRT) for normal listeners is 20 dB sound pressure level or SPL (corresponding to 0 dB hearing loss). The standard time allocated to a subject to respond is approximately four seconds.

The speech discrimination or word recognition is the ability to repeat correctly an open set of monosyllabic words at supra threshold intensity (for example at 60 dB or at 80 dB). The word recognition score (WRS) is the percentage of test words correctly repeated by the subject. The test is administered in a quiet room without any competing noise. Word lists are derived from a standard list with language-specific monosyllabic words. Words are presented randomly to each ear separately, with contralateral masking. They are phonetically balanced, meaning that the speech sounds used occur with the same frequency as in the whole language.

In some embodiments, SSNHL is unilateral. That is to say, in some embodiments, only one ear of the subject in need of treatment is affected by SSNHL. In some embodiments, SSNHL is bilateral. That is to say, in some embodiments, both ears of the subject in need of treatment are affected by SSNHL.

SSNHL may result for example from vestibular schwannoma (acoustic neuroma), stroke, malignancy, vascular ischemia of the inner ear, perilymph fistula, or autoimmune causes (including, without limitation, IgE or IgG allergy). Other causes of SSNHL include for example excessive noise exposure (such as, for example, exposure to a noise of more than about 70, 80, 90, 100, 110, 120, or 130 dB, or more), inner ear involvement by infectious agents (such as, for example, viral and bacterial infections), vasculopathy, ototoxic drugs, head trauma, and blast exposure. However, a cause of SSNHL is identified in only 10 to 15% of subjects.

In some embodiments, SSNHL is idiopathic. That is to say, in some embodiments, no cause of SSNHL was identified despite adequate investigation. In some embodiments, SSNHL is unilateral idiopathic SSNHL.

In some embodiments, SSNHL is noise-induced. Thus, in some embodiments, SSNHL is caused by an acoustic trauma. In some embodiments, SSNHL is caused by an acute acoustic trauma. In some embodiments, SSNHL is unilateral noise-induced SSNHL. In some embodiments, SSNHL is bilateral noise-induced SSNHL.

In some embodiments, severe or profound sudden SSNHL is associated with at least one of:
a hearing threshold at baseline corresponding to a pure tone audiometry (PTA) at baseline equal to or greater than 70 dB, preferably equal to or greater than 80 dB, more preferably equal to or greater than 90 dB;
a hearing loss at baseline equal to or greater than 91 dB corresponding to a profound hearing loss according to the American Speech-Language-Hearing Association (ASHA) classification;

a hearing loss at baseline affecting frequencies equal to or lower than 2000 Hz, in particular ranging from 500 to 1000 Hz; and/or
the presence of vertigo at baseline.

Thus, in other words, in some embodiments, the subject in need of treatment is a subject suffering from severe or profound SSNHL associated with at least one of:
a hearing threshold at baseline corresponding to a PTA at baseline equal to or greater than 70 dB, preferably equal to or greater than 80 dB, more preferably equal to or greater than 90 dB;
a hearing loss at baseline equal to or greater than 91 dB corresponding to a profound hearing loss according to the ASHA classification;
a hearing loss at baseline affecting frequencies equal to or lower than 2000 Hz, in particular ranging from 500 to 1000 Hz; and/or
the presence of vertigo at baseline.

Accordingly, in some embodiments, the present invention relates to azasetron or an analog of azasetron, or a pharmaceutically acceptable salt and/or solvate thereof, for use in the treatment of SSNHL in a subject in need thereof, wherein the subject suffers from severe or profound SSNHL associated with at least one of:
a hearing threshold at baseline corresponding to a PTA at baseline equal to or greater than 70 dB, preferably equal to or greater than 80 dB, more preferably equal to or greater than 90 dB;
a hearing loss at baseline equal to or greater than 91 dB corresponding to a profound hearing loss according to the ASHA classification;
a hearing loss at baseline affecting frequencies equal to or lower than 2000 Hz, in particular ranging from 500 to 1000 Hz; and/or
the presence of vertigo at baseline.

In some embodiments, the present invention relates to azasetron or an analog of azasetron, or a pharmaceutically acceptable salt and/or solvate thereof, for use in the treatment of SSNHL in a subject in need thereof, wherein the subject suffers from severe or profound SSNHL associated with at least one of:
a hearing threshold at baseline corresponding to a PTA at baseline equal to or greater than 80 dB;
a hearing loss at baseline equal to or greater than 91 dB corresponding to a profound hearing loss according to the ASHA classification;
a hearing loss at baseline affecting frequencies equal to or lower than 2000 Hz, in particular ranging from 500 to 1000 Hz; and/or
the presence of vertigo at baseline.

In some embodiments, the present invention relates to azasetron or an analog of azasetron, or a pharmaceutically acceptable salt and/or solvate thereof, for use in the treatment of SSNHL in a subject in need thereof, wherein the subject suffers from severe or profound SSNHL associated with at least one of:
a hearing threshold at baseline corresponding to a PTA at baseline equal to or greater than 90 dB;
a hearing loss at baseline equal to or greater than 91 dB corresponding to a profound hearing loss according to the ASHA classification;
a hearing loss at baseline affecting frequencies equal to or lower than 2000 Hz, in particular ranging from 500 to 1000 Hz; and/or
the presence of vertigo at baseline.

In some embodiments, severe or profound SSNHL is associated with a hearing threshold at baseline corresponding to a PTA at baseline equal to or greater than 70 dB, 80 dB, or 90 dB. Thus, in some embodiments, the subject in need of treatment is a subject suffering from severe or profound SSNHL associated with a hearing threshold at baseline corresponding to a PTA at baseline equal to or greater than 70 dB, 80 dB, or 90 dB.

As indicated above, PTA is a standardized subjective hearing test determining air-conduction and/or bone-conduction hearing thresholds (expressed in dB) at a set of fixed frequencies ranging from 0.25 kHz to 8 kHz. In some embodiments, the PTA is expressed as the average of the hearing threshold (in dB) of the 3 most affected contiguous test frequencies. In some embodiments, the PTA is expressed as the average of the hearing threshold (in dB) of the 3 most affected contiguous test frequencies, using pure tone air-conduction testing.

In some embodiments, severe or profound SSNHL is associated with a hearing loss at baseline equal to or greater than 91 dB corresponding to a profound hearing loss according to the ASHA classification. Thus, in some embodiments, the subject in need of treatment is a subject suffering from severe or profound SSNHL associated with a hearing loss at baseline equal to or greater than 91 dB corresponding to a profound hearing loss according to the ASHA classification.

The ASHA has proposed a classification of hearing loss, depending on the degree of hearing loss, which is commonly used in the field (Clark. ASHA. 1981 July; 23 (7): 493-500). According to the ASHA classification, the degrees of hearing loss are as follows:
hearing loss of −10 to 15 dB: normal,
hearing loss of 16 to 25 dB: slight hearing loss,
hearing loss of 26 to 40 dB: mild hearing loss,
hearing loss of 41 to 55 dB: moderate hearing loss,
hearing loss of 56 to 70 dB: moderately severe hearing loss,
hearing loss of 71 to 90 dB: severe hearing loss,
hearing loss equal to or greater than 91 dB: profound hearing loss.

In some embodiments, the hearing loss at baseline is defined as the difference between the PTA assessed at baseline for the most affected ear and a reference PTA. In some embodiments, preferably when SSNHL is unilateral, the reference PTA is the PTA assessed at baseline for the contralateral ear (i.e., the ear that is not affected or least affected). In some embodiments, the PTA is expressed as the average of the hearing threshold (in dB) of the 3 most affected contiguous test frequencies, preferably using pure tone air-conduction testing. Thus, in some embodiments, the hearing loss at baseline is defined as the difference between the PTA at baseline expressed as the average of the hearing threshold (in dB) of the 3 most affected contiguous test frequencies, preferably using pure tone air-conduction testing, and a reference PTA expressed as the average of the hearing threshold (in dB) of the same frequencies, preferably using pure tone air-conduction testing. In some embodiments, the reference PTA is a PTA previously assessed in the subject (i.e., a PTA previously obtained for the subject). In some embodiments, the reference PTA is a PTA derived from a reference population.

In some embodiments, severe or profound SSNHL is associated with a hearing loss at baseline affecting frequencies equal to or lower than 2000 Hz. Thus, in some embodiments, the subject in need of treatment is a subject suffering from severe or profound SSNHL associated with a hearing loss at baseline affecting frequencies equal to or lower than 2000 Hz.

In some embodiments, severe or profound SSNHL is associated with a hearing loss at baseline affecting frequencies ranging from 500 to 1000 Hz. Thus, in some embodiments, the subject in need of treatment is a subject suffering from severe or profound SSNHL associated with a hearing loss at baseline affecting frequencies ranging from 500 to 1000 Hz.

Humans can detect sounds in a frequency range from about 20 Hz to about 20 kHz. A hearing loss in frequencies below 2000 Hz affects the verbal communication and impacts the quality of life. Subjects with low-frequency hearing loss thus have a hard time hearing low-pitch, deeper sounds, like a man's voice (which is typically deeper than that of a woman or child). They also have difficulties hearing conversation within groups of people and/or hearing in noisy environments and in places with background noise. A hearing loss in frequencies below 2000 Hz also makes it difficult to hear bass sounds when listening to music. A hearing loss affecting frequencies below 2000 Hz is thus considered as a clinical marker of SSNHL severity.

As indicated above, methods for measuring hearing loss are well-known by the skilled artisan. For example, hearing loss may be determined by pure tone audiometry (PTA), which, as detailed above, is a standardized subjective hearing test determining hearing thresholds (expressed in dB) at a set of fixed frequencies ranging from 0.25 kHz to 8 kHz. In some embodiments, the frequencies affected by hearing loss at baseline are thus determined when assessing a PTA at baseline.

In some embodiments, severe or profound SSNHL is associated with the presence of vertigo at baseline. Thus, in some embodiments, the subject in need of treatment is a subject suffering from severe or profound SSNHL associated with the presence of vertigo at baseline.

Methods for assessing the presence of vertigo in a subject are well-known by the skilled artisan. In some embodiments, the presence of vertigo at baseline is assessed on a numerical scale, for example on a numerical scale ranging from 0 to 10, with 0 corresponding to the absence of vertigo and 10 to extreme vertigo.

In some embodiments, severe or profound SSNHL is associated with a hearing threshold at baseline corresponding to a PTA at baseline equal to or greater than 70 dB, 80 dB, or 90 dB; and with a hearing loss at baseline affecting frequencies equal to or lower than 2000 Hz. Thus, in some embodiments, the subject in need of treatment is a subject suffering from severe or profound SSNHL associated with a hearing threshold at baseline corresponding to a PTA at baseline equal to or greater than 70 dB, 80 dB, or 90 dB; and with a hearing loss at baseline affecting frequencies equal to or lower than 2000 Hz.

In some embodiments, severe or profound sudden sensorineural hearing loss (SSNHL) is associated with a hearing threshold at baseline corresponding to a PTA at baseline equal to or greater than 70 dB, 80 dB, or 90 dB; and with a hearing loss at baseline affecting frequencies ranging from 500 to 1000 Hz. Thus, in some embodiments, the subject in need of treatment is a subject suffering from severe or profound SSNHL associated with a hearing threshold at baseline corresponding to a PTA at baseline equal to or greater than 70 dB, 80 dB, or 90 dB; and with a hearing loss at baseline affecting frequencies ranging from 500 to 1000 Hz.

In some embodiments, severe or profound SSNHL is associated with a hearing threshold at baseline corresponding to a PTA at baseline equal to or greater than 70 dB, 80 dB, or 90 dB; and with a hearing loss at baseline equal to or greater than 91 dB corresponding to a profound hearing loss according to the ASHA classification. Thus, in some embodiments, the subject in need of treatment is a subject suffering from severe or profound SSNHL associated with a hearing threshold at baseline corresponding to a PTA at baseline equal to or greater than 70 dB, 80 dB, or 90 dB; and with a hearing loss at baseline equal to or greater than 91 dB corresponding to a profound hearing loss according to the ASHA classification.

In some embodiments, severe or profound SSNHL is associated with a hearing threshold at baseline corresponding to a PTA at baseline equal to or greater than 70 dB, 80 dB, or 90 dB; and with the presence of vertigo at baseline. Thus, in some embodiments, the subject in need of treatment is a subject suffering from severe or profound SSNHL associated with a hearing threshold at baseline corresponding to a PTA at baseline equal to or greater than 70 dB, 80 dB, or 90 dB; and with the presence of vertigo at baseline.

In some embodiments, severe or profound SSNHL is associated with a hearing loss at baseline affecting frequencies equal to or lower than 2000 Hz, in particular frequencies ranging from 500 to 1000 Hz; and with a hearing loss at baseline equal to or greater than 91 dB corresponding to a profound hearing loss according to the ASHA classification. Thus, in some embodiments, the subject in need of treatment is a subject suffering from severe or profound SSNHL associated with a hearing loss at baseline affecting frequencies equal to or lower than 2000 Hz, in particular frequencies ranging from 500 to 1000 Hz; and with a hearing loss at baseline equal to or greater than 91 dB corresponding to a profound hearing loss according to the ASHA classification.

In some embodiments, severe or profound SSNHL is associated with a hearing loss at baseline affecting frequencies equal to or lower than 2000 Hz, in particular frequencies ranging from 500 to 1000 Hz; and with the presence of vertigo at baseline. Thus, in some embodiments, the subject in need of treatment is a subject suffering from severe or profound SSNHL associated with a hearing loss at baseline affecting frequencies equal to or lower than 2000 Hz, in particular frequencies ranging from 500 to 1000 Hz; and with the presence of vertigo at baseline.

In some embodiments, severe or profound SSNHL is associated with a hearing loss at baseline equal to or greater than 91 dB corresponding to a profound hearing loss according to the ASHA classification; and with the presence of vertigo at baseline. Thus, in some embodiments, the subject in need of treatment is a subject suffering from severe or profound SSNHL associated with a hearing loss at baseline equal to or greater than 91 dB corresponding to a profound hearing loss according to the ASHA classification; and with the presence of vertigo at baseline.

In some embodiments, severe or profound SSNHL is associated:
with a hearing threshold at baseline corresponding to a PTA at baseline equal to or greater than 70 dB, 80 dB, or 90 dB;
with a hearing loss at baseline affecting frequencies equal to or lower than 2000 Hz; and
with a hearing loss at baseline equal to or greater than 91 dB corresponding to a profound hearing loss according to the ASHA classification.
   Thus, in some embodiments, the subject in need of treatment is a subject suffering from severe or profound SSNHL associated:
with a hearing threshold at baseline corresponding to a PTA at baseline equal to or greater than 70 dB, 80 dB, or 90 dB;
with a hearing loss at baseline affecting frequencies equal to or lower than 2000 Hz; and
with a hearing loss at baseline equal to or greater than 91 dB corresponding to a profound hearing loss according to the ASHA classification.

In some embodiments, severe or profound SSNHL is associated:
with a hearing threshold at baseline corresponding to a PTA at baseline equal to or greater than 70 dB, 80 dB, or 90 dB;
with a hearing loss at baseline affecting frequencies ranging from 500 to 1000 Hz; and
with a hearing loss at baseline equal to or greater than 91 dB corresponding to a profound hearing loss according to the ASHA classification.
   Thus, in some embodiments, the subject in need of treatment is a subject suffering from severe or profound SSNHL associated:
with a hearing threshold at baseline corresponding to a PTA at baseline equal to or greater than 70 dB, 80 dB, or 90 dB;
with a hearing loss at baseline affecting frequencies ranging from 500 to 1000 Hz;
and with a hearing loss at baseline equal to or greater than 91 dB corresponding to a profound hearing loss according to the ASHA classification.

In some embodiments, severe or profound SSNHL is associated:
with a hearing threshold at baseline corresponding to a PTA at baseline equal to or greater than 70 dB, 80 dB, or 90 dB;
with a hearing loss at baseline affecting frequencies equal to or lower than 2000 Hz; and
with the presence of vertigo at baseline.
   Thus, in some embodiments, the subject in need of treatment is a subject suffering from severe or profound SSNHL associated:
with a hearing threshold at baseline corresponding to a PTA at baseline equal to or greater than 70 dB, 80 dB, or 90 dB;
with a hearing loss at baseline affecting frequencies equal to or lower than 2000 Hz; and
with the presence of vertigo at baseline.

In some embodiments, severe or profound SSNHL is associated:
with a hearing threshold at baseline corresponding to a PTA at baseline equal to or greater than 70 dB, 80 dB, or 90 dB;
with a hearing loss at baseline affecting frequencies ranging from 500 to 1000 Hz; and
with the presence of vertigo at baseline.
   Thus, in some embodiments, the subject in need of treatment is a subject suffering from severe or profound SSNHL associated:
with a hearing threshold at baseline corresponding to a PTA at baseline equal to or greater than 70 dB, 80 dB, or 90 dB;
with a hearing loss at baseline affecting frequencies ranging from 500 to 1000 Hz; and
with the presence of vertigo at baseline.

In some embodiments, severe or profound SSNHL is associated:
with a hearing loss at baseline affecting frequencies equal to or lower than 2000 Hz, in particular frequencies ranging from 500 to 1000 Hz;
with a hearing loss at baseline equal to or greater than 91 dB corresponding to a profound hearing loss according to the ASHA classification; and
with the presence of vertigo at baseline.
   Thus, in some embodiments, the subject in need of treatment is a subject suffering from severe or profound SSNHL associated:

with a hearing loss at baseline affecting frequencies equal to or lower than 2000 Hz, in particular frequencies ranging from 500 to 1000 Hz;
with a hearing loss at baseline equal to or greater than 91 dB corresponding to a profound hearing loss according to the ASHA classification; and with the presence of vertigo at baseline.

In some embodiments, severe or profound SSNHL is associated:
with a hearing threshold at baseline corresponding to a PTA at baseline equal to or greater than 70 dB, 80 dB, or 90 dB;
with a hearing loss at baseline affecting frequencies equal to or lower than 2000 Hz;
with a hearing loss at baseline equal to or greater than 91 dB corresponding to a profound hearing loss according to the ASHA classification; and
with the presence of vertigo at baseline.
   Thus, in some embodiments, the subject in need of treatment is a subject suffering from severe or profound SSNHL associated:
with a hearing threshold at baseline corresponding to a PTA at baseline equal to or greater than 70 dB, 80 dB, or 90 dB;
with a hearing loss at baseline affecting frequencies equal to or lower than 2000 Hz;
with a hearing loss at baseline equal to or greater than 91 dB corresponding to a profound hearing loss according to the ASHA classification; and
with the presence of vertigo at baseline.

In some embodiments, severe or profound SSNHL is associated:
with a hearing threshold at baseline corresponding to a PTA at baseline equal to or greater than 70 dB, 80 dB, or 90 dB;
with a hearing loss at baseline affecting frequencies ranging from 500 to 1000 Hz;
with a hearing loss at baseline equal to or greater than 91 dB corresponding to a profound hearing loss according to the ASHA classification; and
with the presence of vertigo at baseline.
   Thus, in some embodiments, the subject in need of treatment is a subject suffering from severe or profound SSNHL associated:
with a hearing threshold at baseline corresponding to a PTA at baseline equal to or greater than 70 dB, 80 dB, or 90 dB;
with a hearing loss at baseline affecting frequencies ranging from 500 to 1000 Hz;
with a hearing loss at baseline equal to or greater than 91 dB corresponding to a profound hearing loss according to the ASHA classification; and
with the presence of vertigo at baseline.

In some embodiments, the subject in need of treatment is a child, i.e., a subject below the age of 21, 20, 19, or 18. In some embodiments, the subject in need of treatment is an adult, i.e., a subject aged 18, 19, 20, 21 or more. In some embodiments, the subject in need of treatment is at least 30-, 35-, 40-, 45-, 50-, 55-, 60-, 65-70-, 75-, or 80-year-old. In some embodiments, the subject in need of treatment is 30-year-old or older, 40-year-old or older, 50-year-old or older, 60-year-old or older, or 70-year-old or older.

In some embodiments, the subject in need of treatment is a female. In some embodiments, the subject in need of treatment is a male.

In some embodiments, the present invention relates to azasetron, or a pharmaceutically acceptable salt and/or solvate thereof, for use in the treatment of sudden sensorineural hearing loss (SSNHL) in a subject in need thereof as described above, in particular in a subject suffering from severe or profound SSNHL as described above.

Azasetron is a compound of formula (I), wherein * stands for the (R)-enantiomer (or (+)-enantiomer), the (S)-enantiomer (or (−)-enantiomer), the racemate or a non-racemic mixture of (R)- and (S)-enantiomers (corresponding to mixtures of (+)- and (−)-enantiomers):

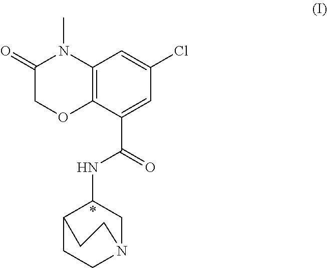 [SMILES: CN1C(=O)COc2c(C(=O)NC3CN4CCC3CC4)cc(Cl)cc21]

Azasetron thus corresponds to 6-chloro-3,4-dihydro-N-(8-methyl-8-azabicyclo-[3.2.1]-oct-3-yl)-2,4-dimethyl-3-oxo-2H-1,4-benzoxazine-8-carboxamide, which may also be referred to as N-(1-azabicyclo[2.2.2]octan-8-yl)-6-chloro-4-methyl-3-oxo-1,4-benzoxazine-8-carboxamide, as described in the U.S. Pat. No. 4,892,872.

As indicated above, azasetron has one chiral center that can give rise to two stereoisomers. In some embodiments, azasetron is racemic azasetron, or a pharmaceutically acceptable salt and/or solvate thereof.

In some embodiments, azasetron is the (R)-enantiomer of azasetron, or a pharmaceutically acceptable salt and/or solvate thereof. The (R)-enantiomer of azasetron is called (R)-azasetron or (+)-azasetron, corresponding to formula (R)-I:

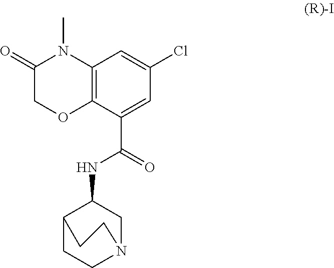 [SMILES: CN1C(=O)COc2c(C(=O)N[C@H]3CN4CCC3CC4)cc(Cl)cc21]

Examples of processes that may be used to synthetize (R)-azasetron or a pharmaceutically acceptable salt and/or solvate thereof are well-known in the art (see for example Chinese patents CN101786963 and CN104557906). In some embodiments, synthesis of (R)-azasetron or of a pharmaceutically acceptable salt and/or solvate thereof includes ab initio synthesis and/or chiral resolution. In some embodiments, when ab initio synthesis of (R)-azasetron or of a pharmaceutically acceptable salt and/or solvate thereof is implemented, at least one racemic starting compound and/or intermediate compound is substituted by a chiral compound.

In some embodiments, azasetron is the (S)-enantiomer of azasetron, or a pharmaceutically acceptable salt and/or solvate thereof. The (S)-enantiomer of azasetron is called (S)-azasetron or (−)-azasetron, corresponding to formula (S)-I:

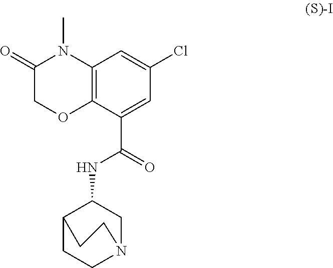 [SMILES: CN1C(=O)COc2c(C(=O)N[C@@H]3CN4CCC3CC4)cc(Cl)cc21]

In some embodiments, azasetron includes the compound of formula I, preferably of formula (R)-I, and all polymorphs, crystals and crystal habits thereof, predrugs and prodrugs thereof, and isotopically-labeled compound of formula I, preferably of formula (R)-I.

Azasetron may be in the form of a pharmaceutically acceptable salt. Pharmaceutically acceptable salts of azasetron include the acid addition salts thereof. Suitable acid addition salts are formed from acids which form non-toxic salts. Examples of acid addition salts include besylate, hydrochloride/chloride, malate, benzoate, ethane-1,2-disulfonate, fumarate, tartrate, acetate, adipate, ascorbate, aspartate, bicarbonate/carbonate, bisulphate/sulphate, borate, camsylate, citrate, cyclamate, edisylate, esylate, ethanesulfonate, formate, gluceptate, gluconate, glucuronate, glutamate, hexafluorophosphate, hibenzate, hydrobromide/bromide, hydroiodide/iodide, isethionate, lactate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, p-toluenesulfonate, tosylate, trifluoroacetate, and xinofoate salts.

Pharmaceutically acceptable salts of azasetron may be prepared by reacting azasetron with the desired acid, or by converting one salt of azasetron to another by reaction with an appropriate acid or by means of a suitable ion exchange column. All these reactions are typically carried out in solution. The salt may precipitate from solution and be collected by filtration or may be recovered by evaporation of the solvent. The degree of ionization in the salt may vary from completely ionized to not ionized resulting in a co-crystal.

In some embodiments, azasetron is in the form of a pharmaceutically acceptable salt selected from a besylate, hydrochloride, malate, benzoate, ethane-1,2-disulfonate, fumarate, and tartrate salt; more preferably from a besylate, hydrochloride, malate, and benzoate salt; even more preferably from a besylate, hydrochloride, and malate salt.

As used herein, “pharmaceutically acceptable solvate” in connection with azasetron refers to a molecular complex comprising azasetron and stoichiometric or sub-stoichiometric amounts of one or more pharmaceutically acceptable solvent molecules such as ethanol or water. The term “hydrate” (as in “pharmaceutically acceptable hydrate”) may be specifically used when the solvent is water.

In some embodiments, azasetron is (R)-azasetron besylate, (R)-azasetron hydrochloride, (R)-azasetron malate, (R)-azasetron benzoate, (R)-azasetron ethane-1,2-disulfonate, (R)-azasetron fumarate, and/or (R)-azasetron tartrate. In some embodiments, azasetron is (R)-azasetron besylate, (R)-azasetron hydrochloride, (R)-azasetron malate, and/or (R)-azasetron benzoate; more preferably azasetron is (R)-azasetron besylate, (R)-azasetron hydrochloride, and/or (R)-azasetron malate.

In some embodiments, azasetron is (R)-azasetron besylate, also referred to as “SENS-401”.

In some embodiments, the present invention relates to an analog of azasetron, or a pharmaceutically acceptable salt and/or solvate thereof, for use in the treatment of sudden sensorineural hearing loss (SSNHL) in a subject in need thereof as described above, in particular in a subject suffering from severe or profound SSNHL as described above.

As used herein, “analog” refers to the modification or substitution of one or more chemical moieties on a parent compound, i.e., azasetron, and may include functional derivatives, positional isomers, tautomers, zwitterions, enantiomers, diastereomers, racemates, isosteres or stereochemical mixtures thereof.

In some embodiments, the analog of azasetron is a benzoxazine compound, or a pharmaceutically acceptable salt and/or solvate thereof. In some embodiments, the analog of azasetron is a benzoxazine compound of the formula (II):

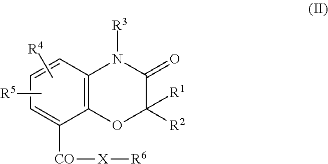

wherein R¹ and R² are the same or different, and each represents hydrogen or C₁₋₈ alkyl; R³ represents hydrogen, C₁₋₈ alkyl, phenylalkyl or substituted phenylalkyl; R⁴ and R⁵ are the same or different, and each represents hydrogen, halogen, C₁₋₈ alkyl, alkoxy, amino, acylamino, C₂₋₅ alkylamino, hydroxy or nitro; X represents oxygen or NH; R⁶ represents a group of the formula (III):

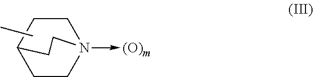

wherein m is 0 or 1,
   or a group of the formula (IV):

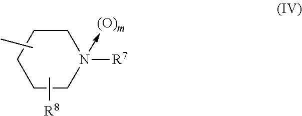

wherein R⁷ represents C₁₋₈ alkyl, phenyl C₁₋₄ alkyl, phenoxyalkyl, substituted phenyl C₁₋₄ alkyl or substituted phenoxyalkyl, R⁸ represents hydrogen or C₁₋₈ alkoxy and m is as defined above,
   or a group of the formula (V):

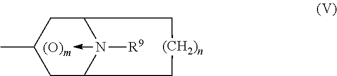

wherein R⁹ represents C₁₋₈ alkyl, phenyl C₁₋₄ alkyl or substituted phenyl C₁₋₄ alkyl, n is 0 or 1, and m is as defined above,
   or a pharmaceutically acceptable salt thereof.

In some embodiments, the analog of azasetron is a benzoxazine compound selected from the group consisting or comprising 6-chloro-3,4-dihydro-2-methyl-3-oxo-N-(3-quinuclidinyl)-2H-1,4-benzoxazine-8-carboxamide, 6-chloro-3,4-dihydro-2,4-dimethyl-3-oxo-N-(3-quinuclidinyl)-2H-benzoxazine-8-carboxamide, 6-chloro-2-ethyl-3,4-dihydro-4-methyl-3-oxo-N-(3-quinuclidinyl)-2H-1,4-benzoxazine-8-carboxamide, 6-chloro-3,4-dihydro-4-methyl-3-oxo-N-(3-quinuclidinyl)-2H-1,4-benzoxazine-8-carboxamide, 6-bromo-3,4-dihydro-2,4-dimethyl-3-oxo-N-(3-quinuclidinyl)-2H-1,4-benzoxazine-8-carboxamide, 6-chloro-3,4-dihydro-2,2,4-trimethyl-3-oxo-N-(3-quinuclidiny-1)-2H-1,4-benzoxazine-8-carboxamide, and pharmaceutically acceptable salts or solvates thereof.

In some embodiments, azasetron or an analog of azasetron, or a pharmaceutically acceptable salt and/or solvate thereof, is to be administered at a therapeutically effective dose.

As used herein, a free base equivalent dose refers to the dose of active ingredient as such, i.e., azasetron or an analog of azasetron, and not to its pharmaceutically acceptable salt and/or solvate form. Thus, compositional variations of a pharmaceutically acceptable salt and/or solvate of azasetron or an analog of azasetron will not impact the free base equivalent dose to be administered.

In some embodiments, azasetron or an analog of azasetron, or a pharmaceutically acceptable salt and/or solvate thereof, is to be administered at a free base equivalent dose ranging from about 0.1 to about 2 mg/kg/day (mg per kilo body weight per day), preferably from about 0.2 to about 1 mg/kg/day. In some embodiments, azasetron or an analog of azasetron, or a pharmaceutically acceptable salt and/or solvate thereof, is to be administered at a free base equivalent dose of about 0.5, 0.6, 0.7, 0.8, 0.9 or 1 mg/kg/day.

In some embodiments, azasetron or an analog of azasetron, or a pharmaceutically acceptable salt and/or solvate thereof, is to be administered at a total daily free base equivalent dose ranging from about 10 to about 500 mg, preferably from about 20 to about 200 mg, more preferably from about 30 to about 100 mg, even more preferably from about 40 to about 60 mg. In some embodiments, azasetron or an analog of azasetron, or a pharmaceutically acceptable salt and/or solvate thereof, is to be administered at a total daily free base equivalent dose of about 20, 30, 40, 50, 60, 70, 80, 90, 100, 110, or 120 mg. In some embodiments, azasetron or an analog of azasetron, or a pharmaceutically acceptable salt and/or solvate thereof, is to be administered at a total daily free base equivalent dose of about 40 mg or of about 60 mg.

In some embodiments, azasetron or an analog of azasetron, or a pharmaceutically acceptable salt and/or solvate thereof, is to be administered at least once a day, preferably twice a day. In some embodiments, azasetron or an analog of azasetron, or a pharmaceutically acceptable salt and/or solvate thereof, is to be administered for at least 1, 2, 3, 4, 5, 6, 7, 8, 9, 10, 11, 12, 13, 14, 15, 16, 17, 18, 19, 20, 21, 22, 23, 24, 25, 26, 27, or 28 days. In some embodiments, azasetron or an analog of azasetron, or a pharmaceutically acceptable salt and/or solvate thereof, is to be administered for at least 1, 2, 3, 4, 5, 6, 7, or 8 weeks. In some embodiments, azasetron or an analog of azasetron, or a pharmaceutically acceptable salt and/or solvate thereof, is to be administered for at least 1, 2, 3, 4, 5, or 6 months.

In some embodiments, azasetron or an analog of azasetron, or a pharmaceutically acceptable salt and/or solvate thereof, is to be administered by injection, orally, topically, transdermally, nasally, by inhalation, buccally, rectally, intratracheally, transmucosally, transtympanically, by percutaneous administration, intramuscularly or by parenteral administration.

In some embodiments, azasetron or an analog of azasetron, or a pharmaceutically acceptable salt and/or solvate thereof, is to be administered systemically.

In some embodiments, azasetron or an analog of azasetron, or a pharmaceutically acceptable salt and/or solvate thereof, is to be administered orally. Thus, in some embodiments, azasetron or an analog of azasetron, or a pharmaceutically acceptable salt and/or solvate thereof, is adapted for oral administration. Examples of formulations adapted for oral administration include: solid forms, liquid forms and gels. Examples of solid forms adapted for oral administration include tablets, pills, capsules, soft gelatine capsules, hard gelatine capsules, caplets, compressed tablets, cachets, wafers, dispersing and/or disintegrating tablets, powders, solid forms suitable for solution, or suspension, in liquid prior to oral administration, and effervescent tablets. Examples of liquid forms adapted for oral administration include solutions, suspensions, drinkable solutions, elixirs, potions, drenches, syrups and liquors.

In some embodiments, azasetron or an analog of azasetron, or a pharmaceutically acceptable salt and/or solvate thereof, alone or in combination with at least one further pharmaceutically active agent, is to be administered as an oral-route form (immediate or sustained-released), preferably as tablets.

In some embodiments, a tablet for oral-route administration comprises or consists of about 1, 2, 3, 4, 5, 6, 7, 8, 9, 10, 11, 12, 13, 14, 15, 16, 17, 18, 19, or 20 mg free base equivalent, preferably about 10 mg free base equivalent, of azasetron or an analog of azasetron, or a pharmaceutically acceptable salt and/or solvate thereof, preferably of (R)-azasetron besylate.

In some embodiments, azasetron or an analog of azasetron, or a pharmaceutically acceptable salt and/or solvate thereof, is to be administered by injection, preferably is to be systemically injected. Thus, in some embodiments, azasetron or an analog of azasetron, or a pharmaceutically acceptable salt and/or solvate thereof, is adapted for injection, in particular systemic injection. Examples of formulations adapted for systemic injections include: liquid solutions or suspensions, solid forms suitable for solution, suspension, in liquid prior to injection. Examples of systemic injections include intravenous, subcutaneous, intramuscular, intradermal, and intraperitoneal injection, or perfusion.

In some embodiments, azasetron or an analog of azasetron, or a pharmaceutically acceptable salt and/or solvate thereof, is to be administered locally.

In some embodiments, azasetron or an analog of azasetron, or a pharmaceutically acceptable salt and/or solvate thereof, is to be topically administered. Thus, in some embodiments, azasetron or an analog of azasetron, or a pharmaceutically acceptable salt and/or solvate thereof, is adapted for topical administration. Examples of formulations adapted for topical administration include sticks, waxes, creams, lotions, ointments, balms, gels, masks, and leave-on washes.

In some embodiments, azasetron or an analog of azasetron, or a pharmaceutically acceptable salt and/or solvate thereof, is to be administered directly in the ear, in particular in the inner ear, middle ear, and/or external ear, for example by transtympanic or intratympanic administration. Thus, in some embodiments, azasetron or an analog of azasetron, or a pharmaceutically acceptable salt and/or solvate thereof, is adapted for administration directly in the ear, in particular in the inner ear, for example by transtympanic or intratympanic administration. Examples of formulations adapted to such administration include otowicks, round window catheters, various types of gels, foams, fibrins, emulsions, solutions, patches or other drug carriers, which are placed in the ear and loaded with azasetron or an analog of azasetron, or a pharmaceutically acceptable salt and/or solvate thereof.

In some embodiments, azasetron or an analog of azasetron, or a pharmaceutically acceptable salt and/or solvate thereof, is to be administered (i.e., first administered) from about 24 hours to about 7 days after the first onset of SSNHL. In some embodiments, azasetron or an analog of azasetron, or a pharmaceutically acceptable salt and/or solvate thereof, is thus to be administered (i.e., first administered) within 1, 2, 3, 4, 5, 6, or 7 days from the first onset of SSNHL. In some embodiments, azasetron or an analog of azasetron, or a pharmaceutically acceptable salt and/or solvate thereof, is to be administered (i.e., first administered) within about 24, 36, 48, 60, 72, 84, or 96 hours from the first onset of SSNHL. In some embodiments, azasetron or an analog of azasetron, or a pharmaceutically acceptable salt and/or solvate thereof, is to be administered (i.e., first administered) within about 96 hours from the first onset of SSNHL.

In some embodiments, azasetron or an analog of azasetron, or a pharmaceutically acceptable salt and/or solvate thereof, is to be administered with at least one further pharmaceutically active agent.

In some embodiments, azasetron or an analog of azasetron, or a pharmaceutically acceptable salt and/or solvate thereof, may be administered simultaneously, separately, or sequentially with said at least one further pharmaceutically active agent.

In some embodiments, azasetron or an analog of azasetron, or a pharmaceutically acceptable salt and/or solvate thereof and the at least one further pharmaceutically active agent are to be administered separately.

In some embodiments, said further pharmaceutically active agent is a corticosteroid. Thus, in some embodiments, azasetron or an analog of azasetron, or a pharmaceutically acceptable salt and/or solvate thereof, is to be administered with at least one corticosteroid. For example, corticosteroids may be selected from oral corticosteroids and/or intra-tympanic corticosteroids. In some embodiments, said corticosteroid is an oral corticosteroid. Examples of oral corticosteroids include dexamethasone, methylprednisolone, prednisone, and prednisolone. In some embodiments, said corticosteroid is an intra-tympanic corticosteroid. Examples of intra-tympanic corticosteroids include dexamethasone, prednisone, hydrocortisone, betamethasone and methylprednisolone.

Another object of the present invention is a method for treating sudden sensorineural hearing loss (SSNHL) in a subject in need thereof as described herein, in particular for treating severe or profound SSNHL in a subject in need thereof as described herein, said method comprising administering to the subject azasetron or an analog of azasetron, or a pharmaceutically acceptable salt and/or solvate thereof.

In some embodiments, the method is for treating SSNHL in a subject suffering from severe or profound SSNHL associated with at least one of:
a hearing threshold at baseline corresponding to a PTA at baseline equal to or greater than 70 dB;
a hearing loss at baseline equal to or greater than 91 dB corresponding to a profound hearing loss according to the ASHA classification;
a hearing loss at baseline affecting frequencies equal to or lower than 2000 Hz; and/or
the presence of vertigo at baseline.

In some embodiments, the method is for treating SSNHL in a subject suffering from severe or profound SSNHL associated with at least one of:
a hearing threshold at baseline corresponding to a PTA at baseline equal to or greater than 80 dB;
a hearing loss at baseline equal to or greater than 91 dB corresponding to a profound hearing loss according to the ASHA classification;
a hearing loss at baseline affecting frequencies equal to or lower than 2000 Hz; and/or
the presence of vertigo at baseline.

In some embodiments, the method is for treating SSNHL in a subject suffering from severe or profound SSNHL associated with at least one of:
a hearing threshold at baseline corresponding to a PTA at baseline equal to or greater than 90 dB;
a hearing loss at baseline equal to or greater than 91 dB corresponding to a profound hearing loss according to the ASHA classification;
a hearing loss at baseline affecting frequencies equal to or lower than 2000 Hz; and/or
the presence of vertigo at baseline.

In some embodiments, the method comprises administering a therapeutically effective amount of azasetron or an analog of azasetron, or a pharmaceutically acceptable salt and/or solvate thereof.

In some embodiments, the method comprises administering at least one further pharmaceutically active agent as described herein. In some embodiments, the method comprises administering at least one corticosteroid as described herein.

In some embodiments, the method further comprises a step of determining that the subject in need of treatment is susceptible to respond to azasetron or an analog of azasetron, or a pharmaceutically acceptable salt and/or solvate thereof.

In some embodiments, the step of determining that the subject in need of treatment is susceptible to respond to azasetron or an analog of azasetron, or a pharmaceutically acceptable salt and/or solvate thereof, comprises assessing that the subject suffers from severe or profound SSNHL as defined herein.

In some embodiments, the step of determining that the subject in need of treatment is susceptible to respond to azasetron or an analog of azasetron, or a pharmaceutically acceptable salt and/or solvate thereof, comprises:
(i) assessing that the subject suffers from severe or profound SSNHL as defined herein; and
(ii) determining that the subject in need of treatment is susceptible to respond to azasetron or an analog of azasetron, or a pharmaceutically acceptable salt and/or solvate, wherein suffering from severe or profound SSNHL as defined herein means that the subject is susceptible to respond to azasetron or an analog of azasetron, or a pharmaceutically acceptable salt and/or solvate.

In some embodiments, assessing that the subject suffers from severe or profound SSNHL comprises assessing the PTA at baseline, the hearing loss at baseline (with regards to the ASHA classification and/or with regards to the affected frequencies), and/or assessing the presence of vertigo as baseline.

Examples of methods for assessing the PTA at baseline, the hearing loss at baseline (with regards to the ASHA classification and/or with regards to the affected frequencies), and for assessing the presence of vertigo as baseline are mentioned herein.

In some embodiments, the presence of at least one of the following:
a hearing threshold at baseline corresponding to a PTA at baseline equal to or greater than 70 dB, 80 dB, or 90 dB;
a hearing loss at baseline equal to or greater than 91 dB corresponding to a profound hearing loss according to the ASHA classification;
a hearing loss at baseline affecting frequencies equal to or lower than 2000 Hz; and/or
the presence of vertigo at baseline,
   means that the subject suffers from severe or profound SSNHL, and thus is susceptible to respond to azasetron or an analog of azasetron, or a pharmaceutically acceptable salt and/or solvate.

Another object of the present invention is a method for treating sudden sensorineural hearing loss (SSNHL) in a subject in need thereof, comprising:
determining that the subject in need of treatment is susceptible to respond to azasetron or an analog of azasetron, or a pharmaceutically acceptable salt and/or solvate thereof, as described above; and
administering azasetron or an analog of azasetron, or a pharmaceutically acceptable salt and/or solvate thereof, to the subject determined to be susceptible to respond to azasetron or an analog of azasetron, or a pharmaceutically acceptable salt and/or solvate thereof.

Another object of the present invention is a pharmaceutical composition for treating or for use in the treatment of sudden sensorineural hearing loss (SSNHL) in a subject in need thereof as described herein, in particular for treating severe or profound SSNHL in a subject in need thereof as described herein, said pharmaceutical composition comprising azasetron or an analog of azasetron, or a pharmaceutically acceptable salt and/or solvate thereof; and optionally at least one pharmaceutically acceptable excipient.

Another object of the present invention is a pharmaceutical composition for treating or for use in the treatment of sudden sensorineural hearing loss (SSNHL) in a subject in need thereof as described herein, in particular for treating severe or profound SSNHL in a subject in need thereof as described herein, said pharmaceutical composition comprising azasetron or an analog of azasetron, or a pharmaceutically acceptable salt and/or solvate thereof.

In some embodiments, the pharmaceutical composition is for administration with at least one further pharmaceutically active agent as described herein. In some embodiments, the pharmaceutical composition is for administration with at least one corticosteroid as described herein.

Another object of the present invention is the use of azasetron or an analog of azasetron, or a pharmaceutically acceptable salt and/or solvate thereof, for the manufacture of a medicament or a kit for the treatment of sudden sensorineural hearing loss (SSNHL) in a subject in need thereof as described herein, in particular for the treatment of severe or profound SSNHL as described herein in a subject in need thereof.

In some embodiments, the medicament or kit is for administration with at least one further pharmaceutically active agent as described herein. In some embodiments, the medicament or kit is for administration with at least one corticosteroid as described herein.

### EXAMPLES

The present invention is further illustrated by the following examples.

### Example

A randomized, double-blind, placebo-controlled, parallel-group phase 2b/3 study was conducted with the aim to assess efficacy and safety of (R)-azasetron in subjects suffering from sudden sensorineural hearing loss. Treatment was administered for 4 weeks (i.e., 28 days) and the study was concluded with an 8-week follow-up at day 84.

Hereinafter, the designation “SENS-401” corresponds to the investigational product (R)-azasetron (administered as (R)-azasetron besylate to the subjects included in the study).

### Materials and Methods

### Subjects Included in the Study

Subjects were included in the study if they presented with sudden sensorineural hearing loss (SSNHL) and met the following criteria:
male or female,
aged at least 18 years old,
subjects with unilateral idiopathic SSNHL or unilateral/bilateral acute acoustic trauma leading to SSNHL defined by both:
   at least one hearing threshold of 50 dB or more amongst the 3 most affected contiguous frequencies on pure tone audiometric air conduction testing performed 24 hours or less prior to first study drug intake, and
   mean hearing loss of 30 dB or more, averaged across the 3 most affected contiguous frequencies on pure tone audiometric air conduction testing performed 24 hours or less prior to first study treatment, compared with the unaffected contralateral ear or reference values from a pre-existing audiogram (dated less than 2 years)
subjects with sudden hearing loss with onset within 96 hours prior to first study drug intake.

As indicated above, hearing loss was determined with pure tone audiometry (PTA) or pure tone audiometric testing, in particular with pure tone audiometric air conduction testing.

Pure tone audiometry (PTA) is widely used to assess SSNHL. The primary purpose of PTA is to determine the degree, type and configuration of hearing loss as its measurement covers the entire auditory pathway from the external ear to the cortex. PTA is a standardized subjective hearing test determining air-conduction and/or bone-conduction hearing thresholds (expressed in decibels or dB) at a set of fixed frequencies ranging from 0.25 kHz to 8 kHz. The hearing thresholds determined at each frequency are plotted on an audiogram for each ear independently.

In the present study, pure tone air- and bone-conduction hearing thresholds were determined for each ear (best ear at first) at 0.25, 0.5, 1, 2, 3, 4, 6, and 8 kHz for air conduction and at 0.5, 1, 2, 3, 4 kHz for bone conduction, using the descending method of limits (modified Hughson Westlake procedure as follows: initial descent towards threshold is accomplished in 10-dB steps. Beginning with the first nonresponse, level is increased by 5-dB for each non-response and decreased by 10-dB after each correct detection response). The hearing threshold was defined as the lowest audible level, measured twice out of three presentations to the subject.

The baseline pure tone audiometry test was defined as the last test performed at site within 24 hours prior to the first study drug administration. The PTA was expressed as the average of the hearing threshold (in dB) of the 3 most affected contiguous test frequencies (air conduction). Therefore, a higher PTA corresponds to a higher hearing threshold and thus to a poorer hearing. Conversely, a lower PTA corresponds to a lower hearing threshold and thus to a better hearing.

The hearing loss was defined as the difference between the PTA expressed as the average of the hearing threshold (in dB) at the 3 most affected contiguous test frequencies (air pure tone average) and the corresponding PTA expressed as the average hearing threshold of the contralateral ear or values from a pre-existing audiogram (dated less than 2 years) for the same frequencies (mandatory for bilateral acute acoustic trauma). The contiguous frequencies used to determine the PTA at baseline remained fixed for all subsequent evaluations. In case of bilateral acute acoustic trauma leading to sudden sensorineural hearing loss, the most affected ear according to the inclusion criteria was considered for the analysis. If both ears were equally affected, the right ear was considered.

Of note, exclusion criteria precluding recruitment in the study included in particular:
bilateral idiopathic hearing loss,
fluctuating hearing loss,
history of asymmetric hearing (>20 dB difference between ears) to the best knowledge of the subject, severe hearing loss (>90 dB) associated with unilateral (ipsilateral) complete vestibular loss,
history of Ménière's disease, autoimmune hearing loss, radiation-induced hearing loss, acoustic neuroma (schwannoma),
previous SSNHL in the affected ear within the past 6 weeks,
history of otosclerosis, suspected perilymph fistula or membrane rupture, suspected retro-cochlear lesion, barotrauma,
congenital or hereditary hearing loss,
history of severe head or neck trauma,
complete loss of peripheral vestibular function on the affected side,
any drug-based therapy for inner ear hearing loss that is ongoing or was performed in the past 6 weeks,
any ongoing or planned concomitant medication for the treatment of tinnitus until 6 weeks after administration,
any therapy known as ototoxic at the current time or in the past 6 months prior to study inclusion,
air-bone gap of greater than 20 dB in 3 contiguous frequencies,
acute chronic otitis media or otitis externa terminated less than 7 days prior to randomization,
history of chronic inflammatory or suppurative ear disease or cholesteatoma,
prior ear surgery of any kind (except ventilating tubes), or cochlear implants except oral corticosteroids.

134 subjects were assessed for eligibility according to the criteria listed above. 115 subjects were randomized with a ratio 1:1:1 in the three following groups (i.e., treatment arms):
38 subjects were included in the group administered for 28 days (i.e., 4 weeks) with placebo,
38 subjects were included in the group administered for 28 days (i.e., 4 weeks) with a “low” dose of (R)-azasetron, defined as a total daily dose of 58 mg of (R)-azasetron (corresponding to a daily dose of 40 mg free base equivalent); and
39 subjects were included in the group administered for 28 days (i.e., 4 weeks) with a “high” dose of (R)-azasetron, defined as a total daily dose of 87 mg of (R)-azasetron (corresponding to a daily dose of 60 mg free base equivalent).

The overall intent-to-treat (ITT) population thus included 115 subjects. However, after taking into consideration the protocol deviations that occurred for some subjects, the overall per-protocol (PP) population included 96 subjects. Of note, a more homogenous overall ITT population of subject suffering from idiopathic SSNHL who received corticoids (also referred to as corticosteroids) between 24 h and 96 h from the onset of the hearing loss included 94 subjects.

### Drug Administration

Subjects were administered placebo or (R)-azasetron (also designated as SENS-401) for 28 days (i.e., 4 weeks). (R)-azasetron was administered orally twice a day either at a dose of 29 mg/BID (bis in die) or at a dose of 43.5 mg/BID (corresponding to a total daily dose of 40 mg and 60 mg free base equivalent, respectively).

In addition to (R)-azasetron or placebo, subjects could be given oral corticosteroids (also referred to as corticoids), such as dexamethasone, methylprednisolone, or prednisone/prednisolone. Thus, a total of 106 subjects received oral corticosteroids (35 subjects in the “placebo” treatment arm, 36 subjects in the “SENS-401 high dose” treatment arm, and 35 subjects in the “SENS-401 low dose” treatment arm). Moreover, in case of non-response, a rescue therapy by intra-tympanic corticosteroids (such as dexamethasone or prednisone) could be administered from day 14. Thus, a total of 14 subjects received intra-tympanic corticosteroids (4 subjects in the “placebo” treatment arm, 4 subjects in the “SENS-401 high dose” treatment arm, and 6 subjects in the “SENS-401 low dose” treatment arm).

### Monitoring of Subjects and Objectives

Subjects included in the study were monitored with a first visit at day −4 to day 0 (screening), followed by a visit at day 1 (D1-randomization and inclusion day), day 7 (D7, ±2 days), day 14 (D14, ±2 days), day 28 (D28±3 days−end of treatment), and day 84 (D84±3 days-follow-up visit marking the end of study). Monitoring included audiometric tests (PTA), speech audiometry, numerical rating scales and questionnaires. Additionally, subjects included in the study were closely monitored for adverse events throughout the study.

The primary objective of the study was to assess the efficacy of SENS-401 (i.e., (R)-azasetron) on hearing loss in comparison to placebo at the end of the 4-week treatment period. Efficacy was assessed by measuring the change in pure tone audiometry or PTA (corresponding to the average of the hearing threshold (in dB) of the 3 most affected contiguous hearing frequencies as identified at study entry) in affected ear from baseline to the end of treatment visit (visit at day 28, ±3 days or D28±3). The change in PTA was also measured in affected ear from baseline to the end of study visit (visit at day 84, ±3 days or D84±3).

As indicated above, a higher PTA corresponds to a higher hearing threshold and thus to a poorer hearing. Conversely, a lower PTA corresponds to a lower hearing threshold and thus to a better hearing. Therefore, a negative change in PTA as compared to the baseline PTA corresponds to a lowering of the hearing threshold and thus to an improvement in hearing. In other words, a lower PTA as compared to the baseline PTA corresponds to a lowering of the hearing threshold and thus to an improvement in hearing.

Additionally, the efficacy of SENS-401 was also assessed by conducting speech audiometry, in particular by conducting a speech discrimination test (also known as word recognition test) to determine a word recognition score (WRS).

Speech discrimination or word recognition is the ability to repeat correctly an open set of monosyllabic words at supra threshold intensity (for example at 60 dB or at 80 dB). The word recognition score (WRS) is thus the percentage of test words correctly repeated by the subject. The test is administered in a quiet room without any competing noise. Word lists are derived from a standard list with language-specific monosyllabic words. Words are presented randomly to each ear separately, with contralateral masking. They are phonetically balanced, meaning that the speech sounds used occur with the same frequency as in the whole language.

A higher word recognition score (WRS) corresponds to a higher percentage of test words correctly repeated by the subject and thus to a better hearing. Conversely, a lower WRS corresponds to a lower percentage of test words correctly repeated by the subject and thus to a poorer hearing. Therefore, a positive change in WRS as compared to the baseline WRS corresponds to an improvement in hearing.

### Statistical Analyses

Mean changes from baseline of the PTA at days 28 and 84 were analyzed using a restricted maximum likelihood (REML)-based repeated measures approach. Analyses included the categorical, fixed covariates of site category (non-ancillary, ancillary), duration of disease and oral corticosteroids intake at baseline (stratification factors) and the fixed, categorical effects of treatment, visit, and treatment-by-visit interaction, as well as the continuous, fixed covariates of baseline score and baseline score-by-visit interaction. An unstructured covariance structure was used to model the within-patient errors. The Kenward-Roger approximation was used to estimate denominator degrees of freedom. The estimations and standard errors of the contrasts of interest were derived from this model. For each timepoint, the marginal predicted means (least squares means) and the corresponding 90% confidence interval were derived for each treatment arm. For each timepoint, 5% one-sided p-values for the comparisons versus placebo were derived. The same approach was applied to the other efficacy endpoints (e.g., speech discrimination test aiming at determining a word recognition score).

### Results

The change in PTA from baseline was assessed by determining the PTA at the end of the treatment (day 28 or D28) and/or at the end of the study (day 84 or D84—follow-up visit). The data are expressed as the mean change in PTA from baseline (dB), corresponding to the difference between the PTA determined at D28 or D84, as indicated, and the PTA at baseline (calculated as PTA at D28 or D84 minus PTA at baseline). As indicated above, a higher PTA corresponds to a higher hearing threshold and thus to a poorer hearing. Conversely, a lower PTA corresponds to a lower hearing threshold and thus to a better hearing. Therefore, a negative change in PTA at D28 or D84 as compared to the baseline PTA (i.e., a negative change in PTA from baseline) corresponds to a lowering of the hearing threshold and thus to an improvement in hearing. In other words, a lower PTA at D28 or D84 as compared to the baseline PTA corresponds to a lowering of the hearing threshold and thus to an improvement in hearing.

Treatment effect (i.e., SENS-401 efficacy) was assessed by determining the difference between treatment arms (ΔPTA SENS-401 vs placebo) calculated as mean change in PTA from baseline (SENS-401) minus mean change in PTA from baseline (placebo). Considering a change in PTA from baseline after treatment with SENS-401 that is negative (indicating a lowering of the hearing threshold and thus an improvement in hearing), a negative ΔPTA indicates a greater PTA decrease from baseline for SENS-401 as compared with placebo. Thus, a negative ΔPTA indicates a beneficial treatment effect of SENS-401 as compared to placebo, translating as an improvement in hearing.

Similarly, the change in word recognition score (WRS) from baseline was assessed by determining the WRS at the end of the treatment (day 28 or D28) and/or at the end of the study (day 84 or D84-follow-up visit). The data are expressed as the mean change in WRS from baseline (%), corresponding to the difference between the WRS determined at D28 or D84, as indicated, and the WRS at baseline (calculated as WRS at D28 or D84 minus WRS at baseline). As indicated above, a higher WRS corresponds to a higher percentage of test words correctly repeated by the subject and thus to a better hearing. Conversely, a lower WRS corresponds to a lower percentage of test words correctly repeated by the subject and thus to a poorer hearing. Therefore, a positive change in WRS at D28 or D84 as compared to the baseline WRS (i.e., a positive change in WRS from baseline) corresponds to an improvement in hearing.

Treatment effect (i.e., SENS-401 efficacy) was assessed by determining the difference between treatment arms (ΔWRS SENS-401 vs placebo) calculated as mean change in WRS from baseline (SENS-401) minus mean change in WRS from baseline (placebo). Considering a change in WRS from baseline after treatment with SENS-401 that is positive (indicating an improvement in hearing), a positive ΔWRS indicates a greater WRS increase from baseline for SENS-401 as compared with placebo. Thus, a positive ΔWRS indicates a beneficial treatment effect of SENS-401 as compared to placebo, translating as an improvement in hearing.

The change in PTA (and/or in WRS) from baseline was determined both in the overall homogenous study population of subjects suffering from idiopathic SSNHL who received corticoids and in specific subpopulations of subjects (suffering from idiopathic SSNHL who received corticoids) defined by clinical markers, in particular clinical markers associated with SSNHL severity. Where indicated, the change in PTA (and/or in WRS) from baseline was determined both in the overall study population and in specific subpopulations of subjects (selected from the overall study population) defined by clinical markers, in particular clinical markers associated with SSNHL severity. Subpopulation analyses were carried out in order to identify the subjects suffering SSNHL most likely to benefit from SENS-401 treatment.

The clinical data detailed below surprisingly show that subjects suffering from severe or profound SSNHL were more susceptible to benefit from treatment with SENS-401 (i.e., (R)-azasetron). Therefore, clinical markers associated with SSNHL severity may be helpful to identify or select subjects suffering from SSNHL most likely to benefit from treatment with (R)-azasetron, such as SSNHL subjects with a hearing threshold at baseline corresponding to a PTA at baseline equal to or greater than 70 dB; SSNHL subjects with a hearing threshold at baseline corresponding to a PTA at baseline equal to or greater than 80 dB; SSNHL subjects with a hearing threshold at baseline corresponding to a PTA at baseline equal to or greater than 90 dB; SSNHL subjects with a hearing loss at baseline equal to or greater than 91 dB (corresponding to a profound hearing loss according to the ASHA classification); SSNHL subjects with a hearing loss at baseline affecting frequencies equal to or lower than 2000 Hz; and/or SSNHL subjects also suffering from vertigo at baseline.

As indicated in Tables 1-3 below, the efficacy of SENS-401 was assessed at day 28 (Table 1) and at day 84 (Tables 2 & 3) in subpopulations of SSNHL subjects selected on their hearing threshold at baseline, corresponding to a PTA at baseline equal to or greater than a specific value.

The PTA at baseline corresponds to the hearing threshold of the affected hear (or the most affected hear), expressed as the average of the hearing threshold (in dB) of the 3 most affected contiguous test frequencies (air-conduction hearing thresholds). As noted above, a higher PTA corresponds to a higher hearing threshold and thus to a poorer hearing.

As indicated in Tables 1 & 2 below, the efficacy of SENS-401 was first assessed in two distinct subpopulations selected from the overall homogenous study population of subjects suffering from idiopathic SSNHL who received corticoids: the subpopulation of subjects having a PTA at baseline equal to or greater than 80 dB (≥80 dB), and the subpopulation of subjects having a PTA at baseline equal to or greater than 90 dB (≥90 dB).

TABLE 1
Comparison of placebo-controlled treatment-effect at day 28 (ΔPTA at
D28) in subpopulations defined using the clinical marker of PTA
at baseline versus overall study population

| | | | Mean change | ΔPTA | |
|---|---|---|---|---|---|
| | | | in | SENS-401 | |
| | | | PTA from | vs placebo | |
| Group | Treatment arm | N | baseline (dB) | (dB) | p-value |
| Overall study | placebo | 28 | −37.9525 | −3.9111 | 0.2754 |
| population | SENS-401 high | 23 | −41.8636 | | |
| PTA ≥ 80 dB | placebo | 15 | −36.6870 | −17.7765 | 0.0442 |
| | SENS-401 high | 9 | −54.4635 | | |
| PTA ≥ 90 dB | placebo | 11 | −30.4120 | −23.2680 | 0.0278 |
| | SENS-401 high | 8 | −53.6800 | | |

Overall study population: ITT overall study population of subjects suffering from idiopathic SSNHL who received corticoids.
PTA: pure tone audiometry.
SENS-401 high: SENS-401 administered at a daily dose of 43.5 mg/BID corresponding to a total daily dose of 60 mg free base equivalent.
ΔPTA SENS-401 vs placebo (dB): difference between treatment arms calculated as mean change in PTA from baseline (SENS-401) minus mean change in PTA from baseline (placebo). A negative ΔPTA indicates a greater PTA decrease from baseline for SENS-401 as compared with placebo.

As shown in Table 1, a significant beneficial effect of SENS-401 administered at a daily dose of 43.5 mg/BID (corresponding to a total daily dose of 60 mg free base equivalent) was observed at day 28 both in the subpopulation of subjects having a PTA at baseline >80 dB, and the subpopulation of subjects having a PTA at baseline ≥90 dB.

Indeed, in the subpopulation of subjects having a PTA at baseline ≥80 dB, the difference between treatment arms (ΔPTA SENS-401 vs placebo) at day 28 was −17.7765 dB (p=0.0442). By comparison, in the overall study population, the difference between treatment arms at day 28 was only of −3.9111 dB (p=0.2754). Of note, at day 28, a beneficial effect of SENS-401 administered at a daily dose of 29 mg/BID (corresponding to a total daily dose of 40 mg free base equivalent) was also observed in the subpopulation of subjects having a PTA at baseline ≥80 dB, with a difference between treatment arms (ΔPTA SENS-401 vs placebo) of −15.2188, greater than that observed in the overall study population (ΔPTA SENS-401 VS placebo=−4.1236).

In the subpopulation of subjects having a PTA at baseline ≥90 dB, the difference between treatment arms (ΔPTA SENS-401 vs placebo) at day 28 was −23.2680 dB (p=0.0278), greater than that observed in the overall study population (ΔPTA SENS-401 vs placebo=−3.9111). Of note, at day 28, a beneficial effect of SENS-401 administered at a daily dose of 29 mg/BID (corresponding to a total daily dose of 40 mg free base equivalent) was also observed in the subpopulation of subjects having a PTA at baseline ≥90 dB, with a difference between treatment arms (ΔPTA SENS-401 vs placebo) of −13.9239, greater than that observed in the overall study population (ΔPTA SENS-401 vs placebo=−4.1236).

TABLE 2
Comparison of placebo-controlled treatment-effect at day 84 (ΔPTA at
D84) in subpopulations defined using the clinical marker of PTA at
baseline versus overall study population (per protocol overall population)

| | | | | ΔPTA | |
|---|---|---|---|---|---|
| | | | Mean change in | SENS-401 | |
| | | | PTA from | vs placebo | |
| Group | Treatment arm | N | baseline (dB) | (dB) | p-value |
| Overall study | placebo | 26 | −37.2177 | −9.0984 | 0.0845 |
| population | SENS-401 high | 18 | −46.3161 | | |
| PTA ≥ 80 dB | placebo | 13 | −35.5651 | −25.6472 | 0.0031 |
| | SENS-401 high | 9 | −61.2123 | | |
| PTA ≥ 90 dB | placebo | 10 | −29.0610 | −28.6788 | 0.0068 |
| | SENS-401 high | 7 | −57.7398 | | |

Overall study population: ITT overall study population of subjects suffering from idiopathic SSNHL who received corticoids.
PTA: pure tone audiometry.
SENS-401 high: SENS-401 administered at a daily dose of 43.5 mg/BID corresponding to a total daily dose of 60 mg free base equivalent.
ΔPTA SENS-401 vs placebo (dB): difference between treatment arms calculated as mean change in PTA from baseline (SENS-401) minus mean change in PTA from baseline (placebo). A negative ΔPTA indicates a greater PTA decrease from baseline for SENS-401 as compared with placebo.

As shown in Table 2, a significant beneficial effect of SENS-401 administered at a daily dose of 43.5 mg/BID (corresponding to a total daily dose of 60 mg free base equivalent) was observed at day 84 both in the subpopulation of subjects having a PTA at baseline ≥80 dB, and the subpopulation of subjects having a PTA at baseline ≥90 dB.

Indeed, in the subpopulation of subjects having a PTA at baseline ≥80 dB, the difference between treatment arms (ΔPTA SENS-401 vs placebo) at day 84 was −25.6472 dB (p=0.0031). By comparison, in the overall study population, the difference between treatment arms at day 84 was only of −9.0984 dB (p=0.0845). Of note, at day 84, a significant beneficial effect of SENS-401 administered at a daily dose of 29 mg/BID (corresponding to a total daily dose of 40 mg free base equivalent) was also observed in the subpopulation of subjects having a PTA at baseline ≥80 dB, with a difference between treatment arms (ΔPTA SENS-401 vs placebo) of −19.4991 (p=0.0161), greater than that observed in the overall study population (ΔPTA SENS-401 vs placebo=−8.7086).

In the subpopulation of subjects having a PTA at baseline ≥90 dB, the difference between treatment arms (ΔPTA SENS-401 vs placebo) at day 84 was −28.6788 dB (p=0.0068), greater than that observed in the overall study population (ΔPTA SENS-401 vs placebo=−9.0984). Of note, at day 84, a beneficial effect of SENS-401 administered at a daily dose of 29 mg/BID (corresponding to a total daily dose of 40 mg free base equivalent) was also observed in the subpopulation of subjects having a PTA at baseline ≥90 dB, with a difference between treatment arms (ΔPTA SENS-401 vs placebo) of −19.0242, greater than that observed in the overall study population (ΔPTA SENS-401 vs placebo=−8.7086).

The efficacy of SENS-401 was also assessed at day 84 in a subpopulation of SSNHL subjects having a PTA at baseline equal to or greater than 70 dB (≥70 dB) selected from the overall study population.

TABLE 3
Comparison of placebo-controlled treatment-effect at day 84 (ΔWRS
at D84) in a subpopulation defined using the clinical marker
of PTA at baseline versus overall study population

| | | | Mean change | | |
|---|---|---|---|---|---|
| | | | in | ΔWRS | |
| | | | WRS from | SENS-401 | |
| Group | Treatment arm | N | baseline (dB) | vs placebo | p-value |
| Overall study | placebo | 22 | 39.4595 | 19.4399 | 0.0303 |
| population | SENS-401 low | 21 | 58.8994 | | |
| PTA ≥ 70 dB | placebo | 14 | 49.9259 | 26.2165 | 0.0428 |
| | SENS-401 low | 13 | 76.1424 | | |

Overall study population: ITT overall study population.
WRS: word recognition score determined at 60 dB.
SENS-401 low: SENS-401 administered at a daily dose of 29 mg/BID corresponding to a total daily dose of 40 mg free base equivalent.
ΔWRS SENS-401 vs placebo (dB): difference between treatment arms calculated as mean change in WRS from baseline (SENS-401) minus mean change in WRS from baseline (placebo). A positive ΔWRS indicates a greater WRS increase from baseline for SENS-401 as compared with placebo.

As shown in Table 3, a significant beneficial effect of SENS-401 was observed at day 84 in the subpopulation of subjects having a PTA at baseline ≥70 dB.

Indeed, when SENS-401 was administered at a daily dose of 29 mg/BID (corresponding to a total daily dose of 40 mg free base equivalent), the difference between treatment arms (ΔWRS SENS-401 vs placebo) at day 84 was 26.2165 (p=0.0428). By comparison, in the overall study population, the difference between treatment arms at day 84 was only of 19.4399 (p=0.0303).

Of note, at day 28, a beneficial effect of SENS-401 administered at a daily dose of 29 mg/BID could also be observed in the subpopulation of subjects having a PTA at baseline ≥70 dB, with a difference between treatment arms (ΔWRS SENS-401 vs placebo) of 12.9673, greater than that observed in the overall study population (ΔWRS SENS-401 vs placebo=8.8241).

As indicated in Table 4 below, the efficacy of SENS-401 was assessed at day 84 in a subpopulation of SSNHL subjects selected on their hearing loss at baseline from the overall homogenous study population of subjects suffering from idiopathic SSNHL who received corticoids. The subjects of the tested subpopulation thus had a hearing loss at baseline equal to or greater than 91 dB (corresponding to a profound hearing loss according to the ASHA (American Speech-Language-Hearing Association) classification).

The hearing loss was defined as the difference between the PTA expressed as the average of the hearing threshold (in dB) at the 3 most affected contiguous test frequencies (air pure tone average) and the corresponding PTA expressed as the average hearing threshold of the contralateral ear or values from a pre-existing audiogram (dated less than 2 years) for the same frequencies (mandatory for bilateral acute acoustic trauma).

The ASHA has proposed a classification of hearing loss, depending on the degree of hearing loss, which is commonly used in the field (Clark. ASHA. 1981 July; 23 (7): 493-500). According to the ASHA classification, the degrees of hearing loss are as follows:
hearing loss of −10 to 15 dB: normal,
hearing loss of 16 to 25 dB: slight hearing loss,
hearing loss of 26 to 40 dB: mild hearing loss,
hearing loss of 41 to 55 dB: moderate hearing loss,
hearing loss of 56 to 70 dB: moderately severe hearing loss,
hearing loss of 71 to 90 dB: severe hearing loss,
hearing loss equal to or greater than 91 dB: profound hearing loss.

TABLE 4
Comparison of placebo-controlled treatment-effect at day 84 (ΔPTA at
D84) in subpopulations defined using the clinical marker of initial
hearing loss at baseline (ASHA definition) versus overall study population

| | | | Mean change | ΔPTA | |
|---|---|---|---|---|---|
| | | | in | SENS-401 | |
| | | | PTA from | vs placebo | |
| Group | Treatment arm | N | baseline (dB) | (dB) | p-value |
| Overall study | placebo | 26 | −37.2177 | −8.7086 | 0.5128 |
| population | SENS-401 low | 22 | −45.9263 | | |
| | placebo | 26 | −37.2177 | −9.0984 | 0.0845 |
| | SENS-401 high | 18 | −46.3161 | | |
| initial hearing | placebo | 5 | −18.6306 | −28.0377 | 0.0428 |
| loss ≥ 91 dB | SENS-401 low | 4 | −46.6683 | | |
| (ASHA | placebo | 5 | −18.6306 | −26.2796 | 0.0367 |
| Profound) | SENS-401 high | 4 | −44.9102 | | |

ASHA: American Speech-Language-Hearing Association.
Overall study population: ITT overall study population of subjects suffering from idiopathic SSNHL who received corticoids.
PTA: pure tone audiometry.
SENS-401 low: SENS-401 administered at a daily dose of 29 mg/BID corresponding to a total daily dose of 40 mg free base equivalent.
SENS-401 high: SENS-401 administered at a daily dose of 43.5 mg/BID corresponding to a total daily dose of 60 mg free base equivalent.
ΔPTA SENS-401 vs placebo (dB): difference between treatment arms calculated as change in PTA from baseline (SENS-401) minus change in PTA from baseline (placebo). A negative ΔPTA indicates a greater PTA decrease from baseline for SENS-401 as compared with placebo.

As shown in Table 4, a significant beneficial effect of SENS-401 was observed at day 84 in the subpopulation of subjects having a hearing loss at baseline (i.e., initial hearing loss) equal to or greater than 91 dB, corresponding to a profound hearing loss according to the ASHA classification.

Indeed, when SENS-401 was administered at a daily dose of 29 mg/BID (corresponding to a total daily dose of 40 mg free base equivalent), the difference between treatment arms (ΔPTA SENS-401 vs placebo) at day 84 was −28.0377 dB (p=0.0428). By comparison, in the overall study population, the difference between treatment arms at day 84 was only of −8.7086 dB (p=0.5128). Similarly, when SENS-401 was administered at a daily dose of 43.5 mg/BID (corresponding to a total daily dose of 60 mg free base equivalent), the difference between treatment arms (ΔPTA SENS-401 vs placebo) at day 84 was −26.2796 dB (p=0.0367). By comparison, in the overall study population, the difference between treatment arms at day 84 was only of −9.0984 dB (p=0.0845).

Of note, at day 28, a beneficial effect of SENS-401 could already be observed in the subpopulation of subjects having a hearing loss at baseline equal to or greater than 91 dB. When SENS-401 was administered at a daily dose of 29 mg/BID, the difference between treatment arms (ΔPTA SENS-401 vs placebo) at day 28 was −19.8922 dB, greater than that observed in the overall study population (ΔPTA SENS-401 vs placebo=−4.1236). Similarly, when SENS-401 was administered at a daily dose of 43.5 mg/BID, the difference between treatment arms (ΔPTA SENS-401 vs placebo) at day 28 was −14.8893, greater than that observed in the overall study population (ΔPTA SENS-401 vs placebo=3.9111).

As indicated in Table 5 below, the efficacy of SENS-401 was assessed at day 84 in a subpopulation of SSNHL subjects selected from the overall study population on the hearing frequencies at baseline that are affected by hearing loss. The efficacy of SENS-401 was thus assessed in an “initial frequency range≤2000 Hz” subpopulation in which the subjects had a hearing loss at baseline affecting frequencies equal to or lower than 2000 Hz (in other words, the subjects of this subpopulation had a hearing loss at baseline affecting a frequency range equal to or lower than 2000 Hz).

Humans can detect sounds in a frequency range from about 20 Hz to about 20 kHz. A hearing loss in frequencies below 2000 Hz affects the verbal communication and impacts the quality of life. Subjects with low-frequency hearing loss thus have a hard time hearing low-pitch, deeper sounds, like a man's voice (which is typically deeper than that of a woman or child). They also have difficulties hearing conversation within groups of people and/or hearing in noisy environments and in places with background noise. A hearing loss in frequencies below 2000 Hz also makes it difficult to hear bass sounds when listening to music. A hearing loss affecting frequencies below 2000 Hz is thus considered as a clinical marker of SSNHL severity.

As indicated above, hearing thresholds are commonly determined by pure tone audiometry (PTA), which is a standardized subjective hearing test determining air-conduction and/or bone-conduction hearing thresholds (expressed in dB) at a set of fixed frequencies ranging from 0.25 kHz to 8 kHz. The PTA is commonly expressed as the average of the hearing threshold (in dB) of the 3 most affected contiguous test frequencies (air-conduction hearing thresholds). The hearing threshold at baseline (corresponding to the PTA at baseline) is thus defined as the hearing threshold of the affected hear (or the most affected hear), expressed as the average of the hearing threshold (in dB) of the 3 most affected contiguous test frequencies (air-conduction hearing thresholds).

TABLE 5
Comparison of placebo-controlled treatment-effect at day 84 (ΔPTA at
D84) subpopulations defined using the clinical marker of initial
frequency range at baseline versus overall study population

| | | | Mean change | ΔPTA | |
|---|---|---|---|---|---|
| | | | in | SENS-401 | |
| | | | PTA from | vs placebo | |
| Group | Treatment arm | N | baseline (dB) | (dB) | p-value |
| Overall study | placebo | 30 | −25.3991 | −7.0694 | 0.1269 |
| population | SENS-401 | 24 | −32.4686 | | |
| | low | | | | |
| Initial frequency | placebo | 22 | −33.0495 | −11.6405 | 0.0483 |
| range ≤ 2000 Hz | SENS-401 | 21 | −44.6900 | | |
| | low | | | | |

Overall study population: ITT overall study population.
PTA: pure tone audiometry.
SENS-401 low: SENS-401 administered at a daily dose of 29 mg/BID corresponding to a daily dose of 40 mg free base equivalent.
ΔPTA SENS-401 vs placebo (dB): difference between treatment arms calculated as change in PTA from baseline (SENS-401) minus change in PTA from baseline (placebo). A negative ΔPTA indicates a greater PTA decrease from baseline for SENS-401 as compared with placebo.

As shown in Table 5, a significant beneficial effect of SENS-401 administered at a daily dose of 29 mg/BID (corresponding to a total daily dose of 40 mg free base equivalent) was observed at day 84 in the subpopulation of subjects having a hearing loss at baseline affecting frequencies ≤2000 Hz.

Indeed, the difference between treatment arms (ΔPTA SENS-401 vs placebo) at day 84 was −11.6405 dB (p=0.0483). By comparison, in the overall study population, the difference between treatment arms at day 84 was only of −7.0694 dB (p=0.1269).

As indicated in Table 6 below, the efficacy of SENS-401 was also assessed in a subpopulation of SSNHL subjects selected on both their hearing threshold at baseline and on the hearing frequencies at baseline that are affected by hearing loss. The subjects of the tested subpopulation, selected from the overall homogenous study population of subjects suffering from idiopathic SSNHL who received corticoids, thus had a PTA at baseline equal to or greater than 80 dB (≥80 dB) and a hearing loss at baseline affecting frequencies equal to or lower than 2000 Hz (initial frequency range≤2000 Hz).

TABLE 6
Comparison of placebo-controlled treatment-effect at day 28 and day
84 (ΔPTA at D28 and at D84) subpopulations defined using the
clinical markers of PTA at baseline & initial frequency range at
baseline versus overall study population

| | | | Mean change | ΔPTA | |
|---|---|---|---|---|---|
| | | | in | SENS-401 | |
| | | | PTA from | vs placebo | |
| Group | Treatment arm | N | baseline (dB) | (dB) | p-value |
| Overall study | placebo | 28 | −37.9525 | −4.1236 | 0.2630 |
| population at | SENS-401 | 26 | −42.0761 | | |
| D28 | low | | | | |
| ≥80 dB & | placebo | 12 | −37.3295 | −21.7623 | 0.0380 |
| ≤2000 Hz at D28 | SENS-401 | 8 | −59.0918 | | |
| | low | | | | |
| Overall study | placebo | 26 | −37.2177 | −8.7086 | 0.5128 |
| population at | SENS-401 | 22 | −45.9263 | | |
| D84 | low | | | | |
| ≥80 dB & | placebo | 11 | −35.0515 | −26.6903 | 0.0050 |
| ≤2000 Hz at D84 | SENS-401 | 8 | −61.7417 | | |
| | low | | | | |

Overall study population: ITT overall study population of subjects suffering from idiopathic SSNHL who received corticoids.
PTA: pure tone audiometry.
SENS-401 low: SENS-401 administered at a daily dose of 29 mg/BID corresponding to a total daily dose of 40 mg free base equivalent.
ΔPTA SENS-401 vs placebo (dB): difference between treatment arms calculated as change in PTA from baseline (SENS-401) minus change in PTA from baseline (placebo). A negative ΔPTA indicates a greater PTA decrease from baseline for SENS-401 as compared with placebo.

As shown in Table 6, a significant beneficial effect of SENS-401 was observed both at day 28 and at day 84 in the subpopulation of subjects having a PTA at baseline equal to or greater than 80 dB and a hearing loss at baseline affecting frequencies equal to or lower than 2000 Hz.

Indeed, when SENS-401 was administered at a daily dose of 29 mg/BID (corresponding to a total daily dose of 40 mg free base equivalent) at day 28, the difference between treatment arms (ΔPTA SENS-401 vs placebo) was −21.7623 dB (p=0.0380). By comparison, in the overall study population, the difference between treatment arms at day 28 was only of −4.1236 dB (p=0.2630). Similarly, when SENS-401 was administered at a daily dose of 29 mg/BID at day 84, the difference between treatment arms (ΔPTA SENS-401 vs placebo) was −26.6903 dB (p=0.0050). By comparison, in the overall study population, the difference between treatment arms at day 84 was only of −8.7086 dB (p=0.5128).

Of note, a beneficial effect of SENS-401 administered at a daily dose of 43.5 mg/BID (corresponding to a total daily dose of 60 mg free base equivalent) could also be observed. At day 84, the difference between treatment arms (ΔPTA SENS-401 vs placebo) was thus-27.9588 (p=0.0030), greater than that observed in the overall study population (ΔPTA SENS-401 vs placebo=−9.0984 (p=0.0845)).

As indicated in Table 7 below, the efficacy of SENS-401 was assessed at day 84 in a subpopulation of SSNHL subjects selected on the presence of vertigo at baseline from the overall homogenous study population of subjects suffering from idiopathic SSNHL who received corticoids.

The presence of vertigo was assessed on a numerical scale ranging from 0 to 10, with 0 corresponding to the absence of vertigo and 10 to extreme vertigo.

TABLE 7
Comparison of placebo-controlled treatment-effect at day 84 (ΔPTA at
D84) in subpopulations defined using the clinical marker presence
of vertigo at baseline versus overall study population

| | | | Mean change | ΔPTA | |
|---|---|---|---|---|---|
| | | | in | SENS-401 | |
| | | | PTA from | vs placebo | |
| Group | Treatment arm | N | baseline (dB) | (dB) | p-value |
| Overall study | placebo | 26 | −37.2177 | −8.7086 | 0.5128 |
| population | SENS-401 low | 22 | −45.9263 | | |
| vertigo at | placebo | 8 | −23.0193 | −21.8040 | 0.0204 |
| baseline | SENS-401 low | 9 | −44.8233 | | |

Overall study population: ITT overall study population of subjects suffering from idiopathic SSNHL who received corticoids.
PTA: pure tone audiometry.
SENS-401 low: SENS-401 administered at a daily dose of 29 mg/BID corresponding to a total daily dose of 40 mg free base equivalent.
ΔPTA SENS-401 vs placebo (dB): difference between treatment arms calculated as change in PTA from baseline (SENS-401) minus change in PTA from baseline (placebo). A negative ΔPTA indicates a greater PTA decrease from baseline for SENS-401 as compared with placebo

As shown in Table 7, a significant beneficial effect of SENS-401 administered at a daily dose of 29 mg/BID (corresponding to a total daily dose of 40 mg free base equivalent) was observed at day 84 in the subpopulation of subjects having vertigo at baseline.

Indeed, the difference between treatment arms (ΔPTA SENS-401 vs placebo) at day 84 was −21.8040 dB (p=0.0204). By comparison, in the overall study population, the difference between treatment arms at day 84 was of −8.7086 (p=0.5128).

Of note, at day 28, a beneficial effect of SENS-401 administered at a daily dose of 29 mg/BID could also be observed in the subpopulation of subjects having vertigo at baseline, with a difference between treatment arms (ΔPTA SENS-401 vs placebo) of −14.0995, greater than that observed in the overall study population (ΔPTA SENS-401 vs placebo=−4.1236).

In the subpopulation of SSNHL subjects selected on the presence of vertigo at baseline, a significant beneficial effect of SENS-401 administered at a daily dose of 29 mg/BID (corresponding to a total daily dose of 40 mg free base equivalent) was also observed at day 84 when assessing the change in WRS from baseline, both at 60 dB and at 80 dB. For example, when assessed at 60 dB, the difference between treatment arms (ΔWRS SENS-401 vs placebo) at day 84 was 53.1273 (p=0.0074). By comparison, in the overall study population, the difference between treatment arms at day 84 was only of 19.4399 (p=0.0303). When assessed at 60 dB, the difference between treatment arms (ΔWRS SENS-401 vs placebo) at day 28 was 35.5287 (p=0.0467). By comparison, in the overall study population, the difference between treatment arms at day 28 was only of 8.8241 (p=0.1948).

1-15. (canceled)

16. A method of treating sudden sensorineural hearing loss (SSNHL) in a subject in need thereof, said method comprising administering azasetron or an analog of azasetron, or a pharmaceutically acceptable salt and/or solvate thereof, to the subject, wherein the subject suffers from severe or profound SSNHL associated with at least one of:
a hearing threshold at baseline corresponding to a pure tone audiometry (PTA) at baseline equal to or greater than 70 dB;
a hearing loss at baseline equal to or greater than 91 dB corresponding to a profound hearing loss according to the American Speech-Language-Hearing Association (ASHA) classification;
a hearing loss at baseline affecting frequencies equal to or lower than 2000 Hz; and/or
a presence of vertigo at baseline.

17. The method according to claim 16, wherein the subject suffers from severe or profound SSNHL associated with a hearing threshold at baseline corresponding to a PTA at baseline equal to or greater than 80 dB.

18. The method according to claim 16, wherein the subject suffers from severe or profound SSNHL associated with a hearing threshold at baseline corresponding to a PTA at baseline equal to or greater than 90 dB.

19. The method according to claim 16, wherein the subject suffers from severe or profound SSNHL associated with a hearing loss at baseline affecting frequencies equal to or lower than 2000 Hz.

20. The method according to claim 16, wherein SSNHL is idiopathic SSNHL.

21. The method according to claim 16, wherein SSNHL is unilateral.

22. The method according to claim 16, wherein azasetron is (R)-azasetron, (S)-azasetron, a mixture thereof, or a pharmaceutically acceptable salt and/or solvate thereof.

23. The method according to claim 16, wherein azasetron is (R)-azasetron or a pharmaceutically acceptable salt and/or solvate thereof.

24. The method according to claim 16, wherein the pharmaceutically acceptable salt is selected from a besylate salt, a malate salt, and a hydrochloride salt.

25. The method according to claim 16, wherein the pharmaceutically acceptable salt of azasetron is (R)-azasetron besylate.

26. The method according to claim 16, wherein the analog of azasetron is a benzoxazine compound or a pharmaceutically acceptable salt and/or solvate thereof.

27. The method according to claim 26, wherein the analog of azasetron is a benzoxazine compound selected from 6-chloro-3,4-dihydro-2-methyl-3-oxo-N-(3-quinuclidinyl)-2H-1,4-benzoxazine-8-carboxamide, 6-chloro-3,4-dihydro-2,4-dimethyl-3-oxo-N-(3-quinuclidinyl)-2H-benzoxazine-8-carboxamide, 6-chloro-2-ethyl-3,4-dihydro-4-methyl-3-oxo-N-(3-quinuclidinyl)-2H-1,4-benzoxazine-8-carboxamide, 6-bromo-3,4-dihydro-2,4-dimethyl-3-oxo-N-(3-quinuclidinyl)-2H-1,4-benzoxazine-8-carboxamide, 6-chloro-3,4-dihydro-2,2,4-trimethyl-3-oxo-N-(3-quinuclidiny-1)-2H-1,4-benzoxazine-8-carboxamide, and pharmaceutically acceptable salts and/or solvates thereof.

28. The method according to claim 16, wherein azasetron or the analog of azasetron, or the pharmaceutically acceptable salt and/or solvate thereof, is administered at a daily dose ranging from about 20 mg to about 200 mg.

29. The method according to claim 16, wherein azasetron or the analog of azasetron, or the pharmaceutically acceptable salt and/or solvate thereof, is administered at a daily dose of about 40 mg or of about 60 mg.

30. The method according to claim 16, wherein azasetron or the analog of azasetron, or the pharmaceutically acceptable salt and/or solvate thereof, is administered within about 96 hours from the first onset of SSNHL.

31. The method according to claim 16, wherein azasetron or the analog of azasetron, or the pharmaceutically acceptable salt and/or solvate thereof, is administered with at least one further pharmaceutically active agent.

32. The method according to claim 31, wherein the at least one further pharmaceutically active agent is a corticosteroid.
